(19) United States
(12) Patent Application Publication
(10) Pub. No.: US 2012/0190752 A1
(43) Pub. Date: Jul. 26, 2012

(54) EXO-S-MECAMYLAMINE METHOD, USE, AND COMPOUND FOR TREATMENT

(75) Inventors: Geoffrey C. Dunbar, Winston-Salem, NC (US); Jessica Beaver, Lewisville, NC (US); Steven M. Toler, Winston-Salem, NC (US)

(73) Assignee: Targacept Inc., Winston-Salem, NC (US)

(21) Appl. No.: 13/383,288

(22) PCT Filed: Jul. 12, 2010
(86) PCT No.: PCT/US10/41685

§ 371 Date: Mar. 27, 2012

Related U.S. Application Data
(60) Provisional application No. 61/225,435, filed on Jul. 14, 2009.
(60) Provisional application No. 61/359,114, filed on Jun. 28, 2010.

(51) Int. Cl. A61K 31/13; A61P 25/18; A61P 25/24

(52) U.S. Cl. 514/661

(57) ABSTRACT

The present invention relates to exo-S-mecamylamine and the use of exo-S-mecamylamine in medical treatments.

### CROSS-REFERENCE TO RELATED APPLICATIONS

This application claims priority to and benefit of U.S. Provisional Application No. 61/225,435, filed Jul. 14, 2009, and U.S. Provisional Application No. 61/359,114, filed Jun. 28, 2010, each herein incorporated by reference.

### FIELD OF THE INVENTION

The present invention relates to exo-S-mecamylamine and the use of exo-S-mecamylamine in medical treatments.

### BACKGROUND OF THE INVENTION

U.S. Pat. No. 7,101,916, herein incorporated by reference, provides for a pharmaceutical composition that includes a therapeutically effective amount of exo-S-mecamylamine or a pharmaceutically acceptable salt thereof, substantially free of exo-R-mecamylamine in combination with a pharmaceutically acceptable carrier. Further, U.S. Pat. No. 7,101,916 provides for the treatment of medical conditions by administering a therapeutically effective amount of exo-S-mecamylamine or a pharmaceutically acceptable salt thereof, substantially free of its exo-R-mecamylamine. The medical conditions include but are not limited to substance addiction (involving nicotine, cocaine, alcohol, amphetamine, opiate, other psychostimulant and a combination thereof), aiding smoking cessation, treating weight gain associated with smoking cessation, hypertension, hypertensive crisis, herpes type I and II, Tourette's Syndrome and other tremors, cancer (such as small cell lung cancer), atherogenic profile, neuropsychiatric disorders (such as bipolar disorder, depression, anxiety disorder, panic disorder, schizophrenia, seizure disorders, Parkinson's disease and attention deficit hyperactivity disorder), chronic fatigue syndrome, Crohn's disease, autonomic dysreflexia, and spasmogenic intestinal disorders.

With an emphasis on the treatment of depression, including Major Depressive Disorder (MDD), the National Institute of Mental Health (NIMH) estimates that approximately 14.8 million American adults suffer from MDD. The Sequenced Treatment Alternatives to Relieve Depression, or STAR*D, study undertaken by NIMH between 2001 and 2006 highlighted the inadequacy of currently available therapies for MDD. Approximately 63% of participants in the study did not achieve remission following initial treatment with an SSRI regimen of citalopram alone. Augmentation therapies may be useful in the treatment of symptoms of depression that do not resolve with first-line treatment. See, Rush, et al., Acute and Longer-Term Outcomes in Depressed Outpatients Requiring One or Several Treatment Steps: A STAR*D Report, American Journal of Psychiatry, November 2006; 163:1905-1917.

U.S. Application Publication No. US 2008/0058345, herein incorporated by reference, provides that mecamylamine or a salt thereof, either in combination or through co-administration with an antidepressant, is particularly useful for treating individuals suffering from major depressive disorder who do not fully respond to conventional therapy, whether the response is partial response or no response. As set forth, a large percentage of patients suffering from a mood disorder, such as major depressive disorder, experience only partial relief of their symptoms or, in fact, do not respond at all. As noted, SSRIs have a level of non-responsiveness estimated about 30%. Treatment with mecamylamine or a salt thereof as an adjunct to the conventional therapy, however, is believed to reduce that gap.

There remains a need for effective treatments of depression, including major depressive disorder, with specific focus toward symptomatic relief including treatment to remission or response, in subjects who are partial or non-responders to conventional therapy.

### SUMMARY OF THE INVENTION

One aspect of the present invention includes a method of reducing one or more symptoms of depression to a subject in need thereof by administering exo-S-mecamylamine substantially free of exo-R-mecamylamine. Similarly, another aspect includes use of exo-S-mecamylamine substantially free of exo-R-mecamylamine in the manufacture of a medicament for reducing one or more symptoms of depression. Similarly, another aspect includes exo-S-mecamylamine substantially free of exo-R-mecamylamine for treatment of one or more symptoms of depression.

Another aspect of the present invention includes a method of eliminating one or more symptoms of depression to a subject in need thereof by administering exo-S-mecamylamine substantially free of exo-R-mecamylamine. Similarly, another aspect includes use of exo-S-mecamylamine substantially free of exo-R-mecamylamine in the manufacture of a medicament for eliminating one or more symptoms of depression. Similarly, another aspect includes exo-S-mecamylamine substantially free of exo-R-mecamylamine for eliminating one or more symptoms of depression.

Another aspect of the present invention includes a method of increasing remission or response rate from one or more symptoms of depression to a subject in need thereof by administering exo-S-mecamylamine substantially free of exo-R-mecamylamine. Similarly, another aspect includes use of exo-S-mecamylamine substantially free of exo-R-mecamylamine in the manufacture of a medicament for increasing remission or response rate from one or more symptoms of depression. Similarly, another aspect includes exo-S-mecamylamine substantially free of exo-R-mecamylamine for increasing remission or response rate from one or more symptoms of depression.

Another aspect of the present invention includes a method of treating one or more symptoms of depression to remission or response to a subject in need thereof by administering exo-S-mecamylamine substantially free of exo-R-mecamylamine. Similarly, another aspect includes use of exo-S-mecamylamine substantially free of exo-R-mecamylamine in the manufacture of a medicament for treating one or more symptoms of depression to remission or response. Similarly, another aspect includes exo-S-mecamylamine substantially free of exo-R-mecamylamine for treatment of one or more symptoms of depression to remission or response.

In certain embodiments of each aspect, the one or more symptoms are related to one or more of: Cognition; Attention; Memory; and Speed of thinking, wherein measurement is made by a Subject Global Impression (Cognition Scale) change from baseline. In certain embodiments of each aspect, the one or more symptom is measured by one or more of HAM-D, Sheehan Disability Scale, or Sheehan Irritability Scale.

Another aspect of the present invention includes a method for improving cognitive function in a depressed subject by administering exo-S-mecamylamine substantially free of exo-R-mecamylamine. Similarly, another aspect includes use of exo-S-mecamylamine substantially free of exo-R-mecamylamine in the manufacture of a medicament for improving cognitive function in a depressed patient. Similarly, another aspect includes exo-S-mecamylamine substantially free of exo-R-mecamylamine for improving cognitive function in a depressed patient.

Another aspect includes a method for decreasing irritability in a subject by administering exo-S-mecamylamine substantially free of exo-R-mecamylamine. Similarly, another aspect includes use of exo-S-mecamylamine substantially free of exo-R-mecamylamine in the manufacture of a medicament for decreasing irritability. Similarly, another aspect includes exo-S-mecamylamine substantially free of exo-R-mecamylamine for decreasing irritability.

Another aspect includes a method for decreasing irritability in a depressed subject by administering exo-S-mecamylamine substantially free of exo-R-mecamylamine. Similarly, another aspect includes use of exo-S-mecamylamine substantially free of exo-R-mecamylamine in the manufacture of a medicament for decreasing irritability in a depressed patient. Similarly, another aspect includes exo-S-mecamylamine substantially free of exo-R-mecamylamine for decreasing irritability in a depressed patient.

In certain embodiments of each aspect, the exo-S-mecamylamine substantially free of exo-R-mecamylamine is administered to patients that are partial responders or non-responders to at least one other treatment. In certain embodiments of each aspect, the at least one other treatment was an anti-depressant or an anti-psychotic used to treat depression. In certain embodiments, the anti-depressant is an SSRI or an SNRI. In certain embodiments of each aspect, the dose of exo-S-mecamylamine substantially free of exo-R-mecamylamine is 1 mg or 2 mg daily. In certain embodiments of each aspect, the rate of onset, namely the amount of time to appreciable effect, is as early as about 2 weeks. In this regard, the time period should not be construed as being exclusive of the onset of effects, which may be earlier, namely in as little as 1 week or 1 day. Rather, certain embodiments of each aspect include effects that manifest within about 2 weeks or less of drug administration. In certain embodiments of each aspect, the exo-S-mecamylamine substantially free of exo-R-mecamylamine maintains a sustained effect of at least 8 weeks. In certain embodiments of each aspect, the administration of exo-S-mecamylamine substantially free of exo-R-mecamylamine provides a higher therapeutic index over conventional therapy. In certain embodiments of each aspect, the administration of exo-S-mecamylamine substantially free of exo-R-mecamylamine provides a higher therapeutic index over first-line therapy. In certain aspect of each embodiment, the subject is diagnosed with depression characterized by one or more of cognitive deficit, attention deficit, irritability, anxiety, disability, decreased quality of life, or memory deficit.

Another aspect of the present invention includes a combination comprising exo-S-mecamylamine substantially free of exo-R-mecamylamine; and one or more antidepressant or antipsychotic. In one embodiment, the combination may occur as separate dosage forms with each active ingredient, administered together or separate, sequentially or concurrently, and close in time or remote in time to each other. Another aspect of the present invention includes a kit comprising: exo-S-mecamylamine substantially free of exo-R-mecamylamine; one or more antidepressant or antipsychotic; and instruction regarding a treatment regimen to treat, delay onset, increase remission or response rate, or delay progression of one or more symptoms of depression. In one embodiment, such a kit may also include packaging, such as a blister pack. Alternatively, such a kit may provide for individual prescription and dosing of each component as separately packaged pharmaceutics, but when combined with the instruction regarding a treatment regimen, such is intended to be within the scope of the present invention. In this regard, the underlying diagnosis of the patient prescribed such treatment need not be of any particular disorder. Rather, the present invention may include symptomatic treatment of one or more of cognitive deficit, attention deficit, irritability, anxiety, disability, decreased quality of life, or memory deficit regardless of disease, disorder, or condition. In one embodiment, the present invention is directed to major depressive disorder, but the present invention should not be limited thereto.

Another aspect of the present invention includes a pharmaceutical composition comprising: exo-S-mecamylamine substantially free of exo-R-mecamylamine; one or more antidepressant or antipsychotic; and one or more pharmaceutically acceptable carrier. In one embodiment, the pharmaceutical composition may be a unitary dosage form. In certain embodiments of each aspect, the antidepressant is an SSRI or an SNRI.

### BRIEF DESCRIPTION OF THE FIGURES

FIG. 1 depicts remission rates (HAM-D≦7) (ITT) as measured by the Hamilton depression rating scale (HAMD≦7). Separation from placebo was seen at week 2.

FIG. 2 depicts subject rated remission (QIDS-SR≦5) rates (ITT N=265) and illustrates remission rates on the QIDS-SR (≦5). Separation from placebo was seen at week 2.

FIG. 3 depicts subject rated response (QIDS-SR≧50%) rates (ITT N=265) and illustrates a response rate on QIDS (50% reduction between baseline and endpoint). Separation was seen at week 1.

### DESCRIPTION OF THE INVENTION

Unless stated otherwise, the following terms and phrases as used herein are intended to have the following meanings. The fact that a particular term or phrase is not specifically defined should not be correlated to indefiniteness or lacking clarity, but rather terms herein are used within their ordinary meaning. When trade names are used herein, applicants intend to independently include the tradename product and the active pharmaceutical ingredient(s) of the tradename product.

The Hamilton Rating Scale for Depression (HRSD), also known as the Hamilton Depression Rating Scale (HDRS) or abbreviated to HAM-D or HAMD, is a multiple choice questionnaire that clinicians may use to rate the severity of a patient's major depression (Hamilton 1967). The questionnaire rates the severity of symptoms observed in depression such as low mood, insomnia, agitation, anxiety and weight loss. The questionnaire is presently one of the most commonly used scales for rating depression in medical research. The clinician chooses the possible responses to each question by interviewing the patient and by observing the patient's symptoms. Each question has multiple possible responses which increase in severity. Although Hamilton's original scale had 17 questions, others later developed HRSD scales with different numbers of questions, the greatest of which is 29 (HRSD-29). Clinicians can use the HRSD in place of, or in conjunction with, the Montgomery-Åsberg Depression Rating Scale (MADRS), the Beck Depression Inventory (BDI), the Zung Self-Rating Depression Scale, the Wechsler Depression Rating Scale, the Raskin Depression Rating Scale, the Inventory of Depressive Symptomatology (IDS), the Quick Inventory of Depressive Symptomatology (QIDS), and other questionnaires.

The Montgomery-Asberg Depression Rating Scale (abbreviated MADRS) is a ten-item diagnostic questionnaire which psychiatrists may use to measure the severity of depressive episodes in patients with mood disorders (Montgomery et al. 1979). It was designed in 1979 by British and Swedish researchers as an adjunct to the Hamilton Rating Scale for Depression (HAMD) which would be more sensitive to the changes brought on by antidepressants and other forms of treatment than the HAMD. There is, however, a high degree of statistical correlation between scores on the two measures.

The Sheehan Disability Scale (SDS) is widely used not only in psychiatry but also in many other chronic medical illnesses because of its generic design. It measures impairment in functioning. The scale generates 4 scores: a work disability score, a social life disability score, a family life disability score and a total score. A total score is generated through addition of the 3 individual scores (work: social life: family life). The maximum possible score is 30.

The 30 item Inventory of Depressive Symptomatology (IDS) (Rush et al. 1986, 1996) and the 16 item Quick Inventory of Depressive Symptomatology (QIDS) (Rush et al. 2003) are designed to assess the severity of depressive symptoms. Both the IDS and the AIDS are available in the clinician (IDS-C₃₀ and QIDS-C₁₆) and self-rated versions (IDS-SR₃₀ and QIDS-SR₁₆). The IDS and QIDS assess all the criterion symptom domains designated by the American Psychiatry Association Diagnostic and Statistical Manual of Mental Disorders—4th edition (DSM-IV) (APA 1994) to diagnose a major depressive episode. These assessments can be used to screen for depression, although they have been used predominantly as measures of symptom severity. The seven day period prior to assessment is the usual time frame for assessing symptom severity. The QIDS-C₃₀ and QIDS-SR₁₆ cover only the nine diagnostic symptom domains used to characterize a major depressive episode, without items to assess atypical, melancholic or their commonly associated symptoms. All 16 items on the QIDS are included within the IDS. The IDS-C₃₀ and IDS-SR₁₆ include the criterion symptoms, as well as commonly associated symptoms (e.g. anxiety, irritability) and items relevant to melancholic, or atypical symptom features. Both versions are sensitive to change, with medications, psychotherapy, or somatic treatments, making them useful for both research and clinical purposes. The psychometric properties of both the IDS and QIDS, have both been established in various study samples. See Rush A J, Trivedi M H, Ibrahim H M, et al., The 16-item Quick Inventory of Depressive Symptomatology (QIDS), Clinician Rating (QIDS-C), and Self-Report (QIDS-SR): a psychometric evaluation in patients with chronic major depression. Biol Psychiatry.; 54: 573-583 (2003).

The Clinical Global Impression rating scales are commonly used measures of symptom severity, treatment response and the efficacy of treatments in treatment studies of patients with mental disorders (Guy, W., 1976). The Clinical Global Impression—Severity scale (CGI-S) is a 7-point scale that requires the clinician to rate the severity of the patient's illness at the time of assessment, relative to the clinician's past experience with patients who have the same diagnosis. Considering total clinical experience, a patient is assessed on severity of mental illness at the time of rating.

The Clinical Global Impression—Improvement scale (CGI-I) is a 7 point scale that requires the clinician to assess how much the patient's illness has improved or worsened relative to a baseline state at the beginning of the intervention. The Clinical Global Impression—Efficacy Index is a 4 point×4 point rating scale that assesses the therapeutic effect of the treatment.

As used herein the acronym SSRI refers to selective serotonin reuptake inhibitor or serotonin-specific reuptake inhibitor, namely a class of compounds class of compounds typically used as antidepressants in the treatment of depression, anxiety disorders, and some personality disorders. SSRIs form a subclass of serotonin uptake inhibitors, which includes other non-selective inhibitors as well. Serotonin-norepinephrine reuptake inhibitors, serotonin-norepinephrine-dopamine reuptake inhibitors and selective serotonin reuptake enhancers are also serotonergic antidepressants. Non-limiting examples of conventional or first-line SSRIs include citalopram (Celexa™, Cipramil™, Cipram™, Dalsan™, Recital™, Emocal™, Sepram™ Seropram™, Citox™); dapoxetine (Priligy™); escitalopram (Lexapro™, Cipralex™, Esertia™); fluoxetine (Prozac™, Fontex™, Seromex™, Seronil™, Sarafem™, Ladose™, Fluctin™ (EUR), Fluox™ (NZ), Depress™ (UZB), Lovan™ (AUS)); fluvoxamine (Luvox™, Fevarin™, Faverin™, Dumyrox™, Favoxil™, Movox™); indalpine (Upstene™) (discontinued); paroxetine (Paxil™ Seroxat™, Sereupin™, Aropax™, Deroxat™, Divarius™, Rexetin™, Xetanor™, Paroxat™ Loxamine™); sertraline (Zoloft™, Lustral™, Serlain™); and zimelidine (Zelmid™, Normud™, (discontinued). Non-limiting examples of SNRIs include venlafaxine (Effexor™); Desvenlafaxine (Pristiq™); Duloxetine (Cymbalta™, Yentreve™ Milnacipran (Dalcipran™, Ixel™, Savella™); Levomilnacipran; Sibutramine (Meridia™, Reductil™); Bicifadine (DOV-220,075); and SEP-227162.

Common conditions with which antipsychotics might be used include schizophrenia, bipolar disorder and delusional disorder. Antipsychotics might also be used to counter psychosis associated with a wide range of other diagnoses, such as depression, including psychotic depression. Further, they may be used as antidepressants, anti-anxiety drugs, mood stabilizers, cognitive enhancers, anti-aggressive, anti-impulsive, anti-suicidal, and hypnotic (sleep) medications. As used herein, a conventional or first-line antipsychotic includes but is not limited to butyrophenones, including haloperidol (Haldol™, Serenace™) and droperidol (Droleptan™); phenothiazines, including chlorpromazine (Thorazine™, Largactil™) fluphenazine (Prolixin™), which is also available in decanoate form, perphenazine (Trilafon™), prochlorperazine (Compazine™), thioridazine (Mellaril™), trifluoperazine (Stelazine™) mesoridazine, periciazine, promazine, triflupromazine (Vesprin™), levomepromazine (Nozinan™), promethazine (Phenergan™), and pimozide (Orap™); thioxanthenes, including chlorprothixene (Cloxan™, Taractan™, Truxal™), clopenthixol (Sordinol™), flupenthixol (Depixol™, Fluanxol™), thiothixene (Navane™), zuclopenthixol (Cisordinol™, Clopixol™ Acuphase™); second generation antipsychotics, which are also referred to as atypical antipsychotics, including clozapine (Clozaril™), olanzapine (Zyprexa™), risperidone (Risperdal™), quetiapine (Seroquel™), ziprasidone (Geodon™), amisulpride (Solian™) asenapine (Saphris™), paliperidone (Invega™), iloperidone (Fanapt™), Zotepine (Nipolept™ Losizopilon™, Lodopin™, Setous™), and sertindole (Serdolect™, and Serlect™ in Mexico); third generation antipsychotics, including aripiprazole (Abilify™), bifeprunox, cannabidiol, tetrabenazine, and metabotropic glutamate receptor 2 agonists, including LY2140023.

Mecamylamine (N,2,3,3-tetramethylbicyclo[2.1.1]heptan-2-amine hydrochloride, 826-39-1) was developed and characterized by Merck & Co., Inc., as a ganglionic blocker with clinically significant hypotensive actions (Stone et al., J Med Pharm Chem 5(4):665-90, 1962). Depending on preferred naming convention, the chemical name for mecamylamine may also be N,2,3,3-tetramethylnorbornan-2-amine. The use of a particular naming convention to generate a chemical name should not affect the scope of the present invention.

Unless otherwise stated herein, the term “mecamylamine” means mecamylamine, its stereoisomers together as the racemic mixture or may also refer to one of the purified separate enantiomers, analogs, the free base, and/or salts thereof. Mecamylamine can be obtained according to the methods and processes described in U.S. Pat. No. 5,986,142, incorporated herein by reference for its teaching regarding method of producing mecamylamine. Purified exo-S-mecamylamine and exo-R-mecamylamine can be obtained according to methods discussed in U.S. Pat. No. 7,101,916, and references cited therein, also incorporated herein by reference for their teaching regarding the production of purified mecamylamine enantiomers. Exo-S-mecamylamine may also be referred to as S-mecamylamine, TC-5214, or (S)—N,2,3,3-tetramethylnorbornan-2-amine, and includes a pharmaceutically acceptable salt thereof.

For reference herein, exo-S-mecamylamine substantially free of exo-R-mecamylamine includes where exo-S-mecamylamine is greater than 95% by weight and exo-R-mecamylamine is less than 5% by weight. More preferably, the substantially pure exo-S-mecamylamine is greater than 98% by weight and exo-R-mecamylamine is less than 2% by weight. More preferably, the substantially pure exo-S-mecamylamine is greater than greater than 99% by weight and exo-R-mecamylamine is less than 1% by weight. Even more preferably, the substantially pure exo-S-mecamylamine is greater than 99.5% by weight and exo-R-mecamylamine is less than 0.5% by weight. Most preferably, the substantially pure exo-s-mecamylamine is greater than 99.7% by weight and exo-R-mecamylamine is less than 0.3% by weight.

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective treatment of a disorder. Treatment of a disorder may be manifested by delaying or preventing the onset or progression of the disorder, as well as the onset or progression of symptoms associated with the disorder. Treatment of a disorder may also be manifested by a decrease or elimination of symptoms, reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. To be administered in an effective dose, compounds may be administered in an amount of as low as about 0.1 mg to about 1000 mg; in certain embodiments, about 0.1 mg to 10 mg; in certain embodiments, about 1 mg to about 5 mg. Thus, an effective dose typically represents the amount that may be administered as a single dose, or as one or more doses that may be administered over a 24 hours period. The dose may be once daily or may be divided so as to provide twice daily (BID), three times a day (QD), four times a day (QID), or more doses. As noted in U.S. Pat. No. 7,101,916, exo-S-mecamylamine may be administered intravenously, intramuscularly, transdermally, intrathecally, orally or by bolus injection. The dosage of exo-S-mecamylamine is about 0.5 mg to about 1000 mg, depending on dosage form exo-S-mecamylamine may be administered one to four times per day. In certain embodiments an effective dose is about 1 mg, about 2 mg, or about 4 mg, as free base equivalents, twice daily, orally.

The present invention includes a salt or solvate of the compounds herein described, including combinations thereof such as a solvate of a salt. The compounds may exist in solvated, for example hydrated, as well as unsolvated forms, and the present invention encompasses all such forms. Typically, but not absolutely, the salts of the present invention are pharmaceutically acceptable salts. Salts encompassed within the term “pharmaceutically acceptable salts” refer to non-toxic salts of the compounds of this invention. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. In certain embodiments, S-mecamylamine hydrochloride is a preferential salt form.

Although it is possible to administer the compound of the present invention in the form of a bulk active chemical, it is preferred to administer the compound in the form of a pharmaceutical composition or formulation. Thus, one aspect the present invention includes pharmaceutical compositions comprising one or more compounds of Formula I and/or pharmaceutically acceptable salts thereof and one or more pharmaceutically acceptable carriers, diluents, or excipients. Another aspect of the invention provides a process for the preparation of a pharmaceutical composition including admixing one or more compounds of Formula I and/or pharmaceutically acceptable salts thereof with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

One embodiment of an oral pharmaceutical composition includes about 0.6 mg S-mecamylamine hydrochloride; about 6.1 mg microcrystalline cellulose, grade I; about 102.5 mg microcrystalline cellulose, grade II; about 6.0 mg hydroxypropyl cellulose; about 3.6 mg croscarmellose sodium; about 0.6 mg colloidal silicon dioxide; and about 0.6 mg magnesium. One embodiment of a pharmaceutical composition includes about 1.2 mg S-mecamylamine hydrochloride; about 12.2 mg microcrystalline cellulose, grade I; about 95.8 mg microcrystalline cellulose, grade II; about 6.0 mg hydroxypropyl cellulose; about 3.6 mg croscarmellose sodium; about 0.6 mg colloidal silicon dioxide; and about 0.6 mg magnesium. One embodiment of a pharmaceutical composition includes about 2.4 mg S-mecamylamine hydrochloride; about 24.4 mg microcrystalline cellulose, grade I; about 82.4 mg microcrystalline cellulose, grade II; about 6.0 mg hydroxypropyl cellulose; about 3.6 mg croscarmellose sodium; about 0.6 mg colloidal silicon dioxide; and about 0.6 mg magnesium. One embodiment of a pharmaceutical composition includes about 4.9 mg S-mecamylamine hydrochloride; about 25.0 mg microcrystalline cellulose, grade I; about 79.3 mg microcrystalline cellulose, grade II; about 6.0 mg hydroxypropyl cellulose; about 3.6 mg croscarmellose sodium; about 0.6 mg colloidal silicon dioxide; and about 0.6 mg magnesium. One embodiment for manufacture includes blending and sieving the excipients as is known in the art.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses. The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

Exo-S-mecamylamine may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, prednisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time. Another aspect of the present invention includes combination therapy comprising administering to the subject a therapeutically or prophylactically effective amount of the compound of the present invention and one or more other therapy including chemotherapy, radiation therapy, gene therapy, or immunotherapy.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C— or ¹⁴C-enriched carbon are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

### EXAMPLES

A Phase 2b clinical trial of exo-S-mecamylamine as an augmentation (add-on) treatment for major depressive disorder, or MDD, in subjects who did not respond adequately to first-line treatment with citalopram alone was conducted. The result on the primary outcome measure for the trial, mean change between TC-5214 and placebo from baseline on the Hamilton Rating Scale for Depression-17, or HAM-D, was highly statistically significant in favor of TC-5214 (p<0.0001) on an intent to treat basis. The results on all of the trial's secondary efficacy measures, including assessments of depression, irritability, disability, cognition, severity of illness and global improvement, were also statistically significant in favor of TC-5214 on an intent to treat basis.

The Phase 2b trial of TC-5214 as an augmentation treatment for MDD was a two-phase trial conducted at 20 sites in India and three sites in the United States. In the first phase, 579 subjects with MDD received first-line treatment with citalopram hydrobromide for eight weeks, 20 mg daily for the first four weeks and 40 mg daily for the next four weeks. Citalopram, an approved treatment for MDD marketed in the United States as Celexa®, is from the drug class known as selective serotonin reuptake inhibitors. At the end of the eight weeks, subjects whose MADRS score had improved less than 50 percent and whose CGI-SI score was no lower than 4 were considered partial or non responders and randomized into the double blind second phase of the trial.

In the double blind second phase, subjects continued their citalopram treatment and also received either add-on TC-5214 or add-on placebo for an additional eight weeks. The daily dosage of TC-5214 was initially 2 mg and could be increased at the discretion of the investigator to 4 mg and to 8 mg based on tolerability and therapeutic response.

The primary outcome measure for the trial was mean change between TC-5214 and placebo from double blind baseline as measured by HAM-D at week 16. The intent to treat dataset included 265 subjects in the second phase.

In more detail, a multi-center double-blind, randomized, placebo-controlled, parallel group, flexible dose titration study of TC-5214 as adjunctive therapy in subjects with major depressive disorder (MDD) who were inadequate responders to citalopram was conducted in centers in the US and India. The study consisted of a screening period, baseline/washout period, open-label phase, double-blind phase, and a follow-up visit. Subject participation in the study could continue for up to 16 weeks of treatment. Following a washout period of up to 28 days for other antidepressants, subjects with MDD and a MADRS total score >/=28 and a Clinical Global Impression-Severity (CGI-S) score >/=4 were enrolled on Day 1 into an open-label citalopram-only phase of the study. This phase lasted 8 weeks, with citalopram (CIT) dose increased from a once-daily dose of 20 mg (Day 1 through Week 4) to 40 mg (Week 4 through Week 8). A total of 579 subjects entered the open-label citalopram phase. At Week 8, subjects who tolerated 40 mg CIT, but whose MADRS score was reduced <50% from baseline and no lower than 17, and CGI-S≧4, were considered inadequate responders. These subjects (n=270) were randomized in a double-blind fashion to receive either placebo or TC-5214-23 as add-on therapy to continued CIT. The double-blind phase of the study also lasted 8 weeks (Week 8 to Week 16). Study drug, TC-5214-23 or placebo, was started at 2 mg daily (1 mg twice per day [BID] dosing), added on to continuing CIT (40 mg PO qd). After 2 weeks of treatment, the dose of TC-5214 could be increased to 4 mg (2 mg BID) or continued unchanged, with up-titration based upon good tolerability and inadequate therapeutic response. After a further 2 weeks, the dose of TC-5214 could be increased again to 8 mg (4 mg BID) if judged appropriate by the investigator, based upon good tolerability and inadequate therapeutic response. At any time during the double-blind phase of the study, placebo or TC-5214-23 could be reduced to the previous dose level following the emergence of unacceptable adverse events (AEs). Subjects who did not tolerate 2 mg were withdrawn from the study. Subjects who completed the double-blind phase of the study (Week 16) had a follow-up visit 2 to 3 weeks after the last dose of trial medication. At this follow-up, any signs or symptoms of relapse were evaluated. If a subject was prematurely discontinued from the study between Week 8 and Week 16 for any reason, the investigator was to make every effort to perform all evaluations as per protocol, assuming the subject had reached the end of the double-blind add-on treatment phase. These evaluations were to be made as soon as possible and within 2 weeks of discontinuation. The mean difference between Week 8 and Week 16 LOCF in efficacy outcome measures was statistically significant favoring TC-5214-23 for all primary (HAMD-17 total score; P<0.0001) and secondary endpoints (MADRS total score; Quick Inventory of Depressive Symptomatology-Self Reported [QIDS-SR]; Clinical Global Impression-Severity of Illness (CGI-S); CGI-Global Improvement [CGI-G1]; Sheehan Irritability Scale [SIS] Score; Sheehan Disability Scale [SDS] score; and Subject Global Impression-Cognition [SGI-Cog] scale total score with its 3 SGI-Cog subscale scores [Memory, Attention, and Speed of Thinking]: all P<0.0001). There was no meaningful difference in results as a function of age, gender, site, or smoking status. Tables 1 and 2 list the primary and secondary efficacy endpoint results for the ITT population.

TABLE 1
Primary and Secondary Efficacy Endpoint Results

| | Placebo + CIT | | TC-5214 + CIT | | Difference | | |
|---|---|---|---|---|---|---|---|
| | Adj. Mean (SE) | | Adj. Mean (SE) | | (95% Confidence | | |
| Parameter | (N = 132) | | (N = 133) | | Interval) | | P-value |
| HAMD-17 | −7.75 | (0.62) | −13.75 | (0.62) | −6.0 | (−7.72, −4.27) | <0.0001 |
| MADRS | −9.82 | (0.88) | −17.26 | (0.88) | −7.45 | (−9.88, −5.01) | <0.0001 |
| QIDS-SR | −4.31 | (0.42) | −8.07 | (0.41) | −3.76 | (−4.91, −2.61) | <0.0001 |
| CGI-SI | −0.92 | (0.10) | −1.79 | (0.10) | −0.87 | (−1.13, −0.60) | <0.0001 |
| CGI-GI | 2.70 | (0.09) | 1.91 | (0.09) | −0.79 | (−1.04, 0.54) | <0.0001 |
| SDS | −4.69 | (0.53) | −9.18 | (0.53) | −4.49 | (−5.96, −3.01) | <0.0001 |
| SIS | −9.66 | (1.12) | −18.21 | (1.12) | −8.54 | (−11.65, −5.44) | <0.0001 |
| SGI-Cognition | 8.66 | (0.27) | 6.52 | (0.27) | −2.15 | (−2.89, −1.14) | <0.0001 |
| SGI-Attention | 2.85 | (0.09) | 2.13 | (0.09) | −0.72 | (−0.96, 0.47) | <0.0001 |
| SGI-Memory | 2.90 | (0.09) | 2.23 | (0.09) | −0.67 | (−0.93, −0.42) | <0.0001 |
| SGI-Speed | 2.92 | (0.09) | 2.16 | (0.09) | −0.76 | (−1.02, −0.50) | <0.0001 |

TABLE 2
Efficacy Results (ITT N = 265)

| | Adj. Mean | Adj. Mean | Difference | |
|---|---|---|---|---|
| | (SE) PBO | (SE) TC-5214 | (Confidence | |
| Parameter | (n = 132) | (n = 133 | Interval) | P-value |
| SDS | −4.69 (0.53) | −9.18 (0.53) | −4.49 (−5.96, −3.01) | <0.0001 |
| SIS | −9.66 (1.12) | −18.21 (1.12) | −8.54 (−11.65, −5.44) | <0.0001 |
| SGI-Cognition | 8.66 (0.27) | 6.52 (0.27) | −2.15 (−2.89, −1.41) | <0.0001 |
| SGI-Attention | 2.85 (0.09) | 2.13 (0.09) | −0.72 (−0.96, −0.47) | <0.0001 |
| SGI-Memory | 2.90 (0.09) | 2.23 (0.09) | −0.67 (−0.93, −0.42) | <0.0001 |
| SGI-Speed | 2.92 (0.09) | 2.16 (0.09) | −0.76 (−1.02, −0.50) | <0.0001 |

The above results are based on GLM with baseline score as a covariate. No other covariates were used.
SDS = Sheehan Disability Scale
SIS = Sheehan Irritability Scale. This was measured at baseline and endpoint. Any change was calculated.
SGI-Cognition. This is the SUM of the next 3 items. It was assessed at endpoint and the patient rated change from baseline.
SGI = Subject Global Impression
SGI Attention. Measures subjective change in attention/concentration
SGI Memory. Measures subjective change in memory and learning.
SGI Speed. Measures subjective speed of thoughts/thinking.

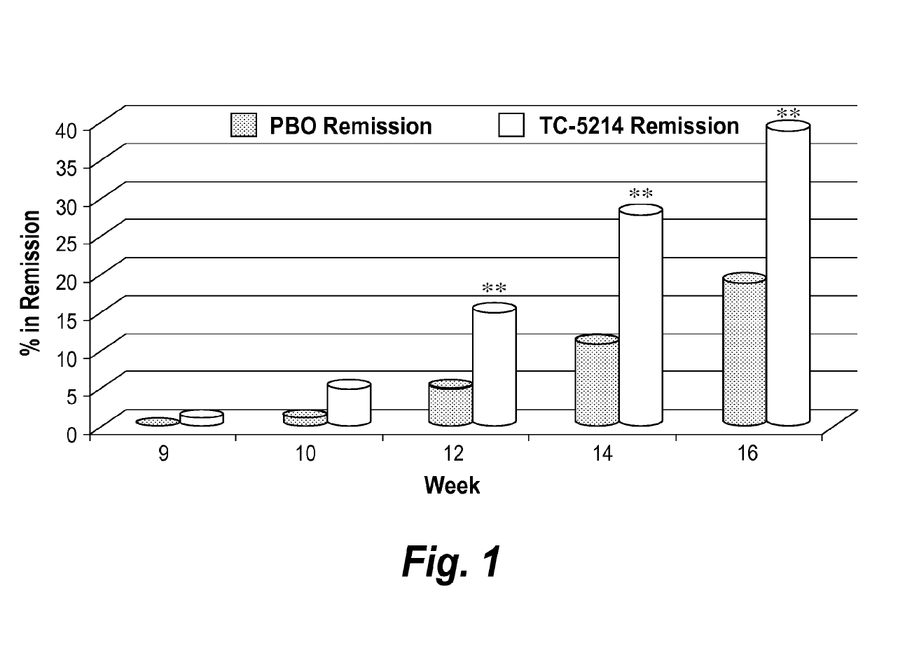

As shown in FIG. 1, remission rates were measured by the Hamilton depression rating scale and provided scores of HAMD≦7. A separation from placebo, namely the onset of action, was observed on or before week 2 for the remission or response rate from one or more symptoms of depression by administering exo-S-mecamylamine substantially free of exo-R-mecamylamine.

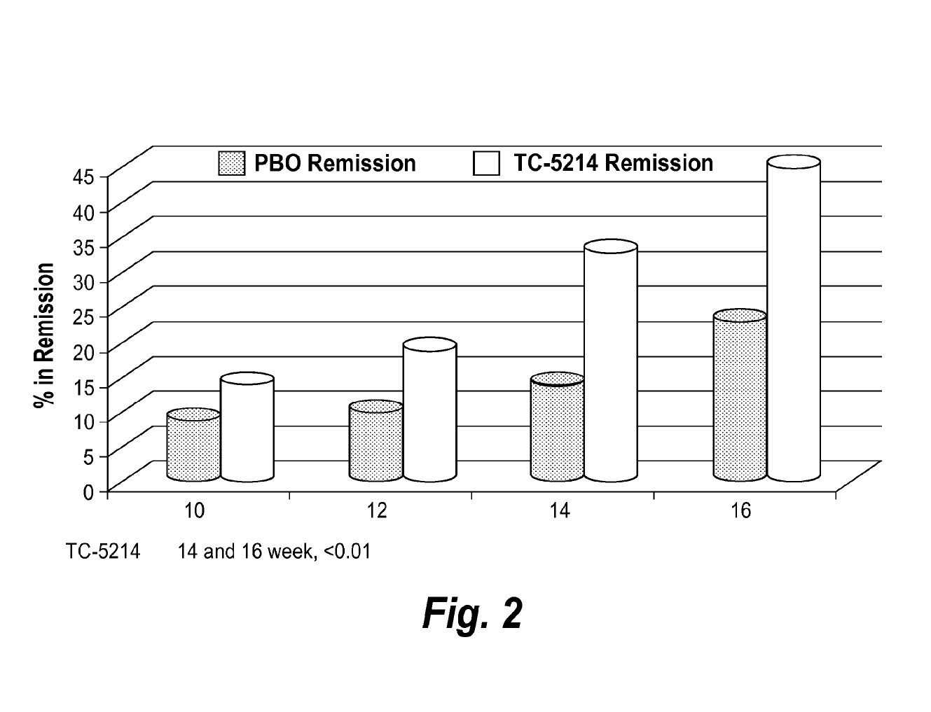

FIG. 2 depicts subject rated remission (QIDS-SR≦5) rates (ITT N=265) and illustrates remission rates on the QIDS-SR (≦5). A separation from placebo, namely, again, that the effects of the exo-S-mecamylamine manifest, was observed on or before week 2 to provide remission or response.

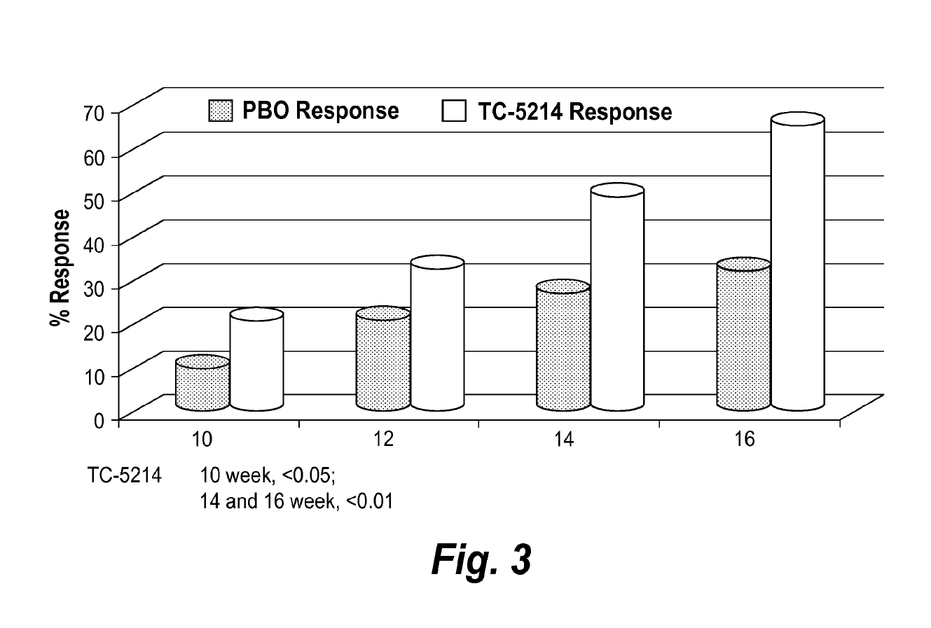

FIG. 3 depicts subject rated response (QIDS-SR≧50%) rates (ITT N=265) and illustrates a response rate on QIDS (50% reduction between baseline and endpoint). A separation from placebo, namely, again, that the effects of the exo-S-mecamylamine manifest, was observed on or before week 1 to provide remission or response.

With regard to efficacy endpoints, the following tables present efficacy used in the ITT (Intent-to-Treat) and PP (Per Protocol) populations unless otherwise specific. The primary inference was based upon Week 16 visit using the ITT population. Change from baseline was obtained by subtracting the baseline values from the individual on-treatment values. Change from Week 16 was obtained by subtracting the Week 16 values from the individual follow-up visit (Week 18/19) values. If either the baseline or Week 16 or on-treatment or follow-up value was missing, the change from baseline or Week 16 value was also set to missing. To account for missing data, the last observation carry forward (LOCF) method was used for the ITT and PP populations. Thus, the last available on-therapy observation for a subject, including the observation at premature discontinuation, was used to estimate subsequent missing data points. For efficacy, baseline data were collected at Week 8 in the double-blind augmentation phase electronic case report form pages.

Table 3 presents a summary of observed scores for the HAMD-17 at Week 8, Week 16, and Week 18 for the ITT and PP populations.

TABLE 3

| | HAMD-17 TOTAL SCORE | | | | | | | |
|---|---|---|---|---|---|---|---|---|
| | Treatment | N | Mean | StdDev | StdErr | Median | Min | Max |
| ITT Population | | | | | | | | |
| Double Blind Week 8 (Baseline) | Placebo | 132 | 23.7 | 4.68 | 0.41 | 24.0 | 12 | 34 |
| | TC-5214 | 133 | 23.5 | 5.19 | 0.45 | 24.0 | 11 | 37 |
| Double Blind Week 16 LOCF | Placebo | 132 | 15.9 | 8.14 | 0.71 | 15.0 | 0 | 35 |
| | TC-5214 | 133 | 9.8 | 6.68 | 0.58 | 8.0 | 0 | 31 |
| Double Blind Week 18 LOCF | Placebo | 132 | 14.7 | 7.84 | 0.68 | 14.5 | 2 | 34 |
| (Follow-up) | TC-5214 | 133 | 9.4 | 6.58 | 0.57 | 8.0 | 0 | 29 |
| PP Population | | | | | | | | |
| Double Blind Week 8 (Baseline) | Placebo | 112 | 23.8 | 4.70 | 0.44 | 25.0 | 12 | 34 |
| | TC-5214 | 113 | 23.7 | 5.20 | 0.49 | 24.0 | 11 | 37 |
| Double Blind Week 16 LOCF | Placebo | 112 | 15.2 | 8.06 | 0.76 | 14.0 | 0 | 33 |
| | TC-5214 | 113 | 9.1 | 6.44 | 0.61 | 8.0 | 0 | 31 |
| Double Blind Week 18 LOCF | Placebo | 112 | 14.0 | 7.72 | 0.73 | 13.5 | 2 | 29 |
| (Follow-up) | TC-5214 | 113 | 8.6 | 6.24 | 0.59 | 7.0 | 0 | 29 |

Table 4 presents primary efficacy analysis results for the HAMD-17 for the ITT and PP populations.

TABLE 4

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −7.75 | 0.62 | | | | |
| | TC-5214 | −13.75 | 0.62 | | | | |
| | | | | 6.00 | 4.27 | 7.72 | <0.0001 |
| Double Blind Week 18 LOCF | Placebo | −0.83 | 0.25 | | | | |
| (Follow-up) | TC-5214 | −0.70 | 0.25 | | | | |
| | | | | −0.13 | −0.86 | 0.59 | 0.7191 |
| PP Population | | | | | | | |
| Double Blind Week 16 LOCF | Placebo | −8.54 | 0.67 | | | | |
| | TC-5214 | −14.64 | 0.66 | | | | |
| | | | | 6.10 | 4.25 | 7.95 | <0.0001 |
| Double Blind Week 18 LOCF | Placebo | −0.83 | 0.29 | | | | |
| (Follow-up) | TC-5214 | −0.83 | 0.29 | | | | |
| | | | | −0.01 | −0.84 | 0.83 | 0.9901 |

Definitions: LOCF = Last Observation Carried Forward; CI = Confidence Interval

The TC-5214 group had statistically significant decreases (improvements) from baseline in HAMD-17 total score compared with placebo for both the ITT and PP populations.

Analysis for the secondary endpoints were performed on both the ITT and PP populations. The secondary endpoints were as follows in Table 5.

TABLE 5

| Variable | Endpoint |
|---|---|
| QIDS-SR | change from Week 8 (double-blind baseline) |
| | to Week 16 |
| HAMD Anxiety/ | change from Week 8 (double-blind baseline) |
| Somatization Factor | to Week 16 |
| MADRS | change from Week 8 (double-blind baseline) |
| | to Week 16 |
| MADRS Proportion | proportion of subjects assessed to be in |
| of Remission | remission as defined by a MADRS score |
| | of ≦10 or ≦12 at Week 16 |
| MADRS Proportion | proportion of subjects assessed to be |
| of Responders | responders as defined by a MADRS reduction |
| | from baseline ≧50% |
| HAMD-17 Proportion | proportion of subjects assessed to be in |
| of Remission | remission as defined by a HAMD-17 score |
| | of ≦7 or ≦10 at Week 16 |
| HAMD-17 Proportion | proportion of subjects assessed to be |
| of Responders | responders, as defined by a HAMD-17 |
| | reduction from baseline ≧50% |
| QIDS Proportion | proportion of subjects assessed to be in |
| of Remission | remission as defined by a QIDS score |
| | of ≦5 at Week 16 |
| QIDS Proportion | proportion of subjects assessed to be |
| of Responders | responders as defined by a QIDS reduction |
| | from baseline ≧50%. |
| CGI-SI | change from Week 8 (double-blind baseline) |
| | to Week 16 |
| CGI-I | change from Week 8 (double-blind baseline) |
| | to Week 16 |
| CGI Efficacy | proportion of subjects in each treatment |
| Index | group whose therapeutic benefit by the CGI |
| | Global Efficacy Index outweighed their |
| | adverse effects at each visit |
| SDS | change from Week 8 (double-blind baseline) |
| | to Week 16 |
| SIS | change from Week 8 (double-blind baseline) |
| | to Week 16 |
| SGI-Cog | Week 16 results for the SGI-Cog. |

With regard to QIDS-SR, as shown in Table 6, the TC-5214 group had statistically significant decreases (improvements) from baseline in QIDS-SR score compared with the placebo group for both the ITT and PP populations. The TC-5214 group also had a statistically significant decrease at Week 18 in QIDS-SR score compared with placebo group for the PP population.

TABLE 6

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −4.28 | 0.42 | | | | |
| | TC-5214 | −8.07 | 0.42 | | | | |
| | | | | 3.79 | 2.63 | 4.94 | <0.0001 |
| Double Blind Week 18 LOCF | Placebo | 0.05 | 0.25 | | | | |
| (Follow-up) | TC-5214 | −0.52 | 0.25 | | | | |
| | | | | 0.58 | −0.13 | 1.28 | 0.1112 |
| PP Population | | | | | | | |
| Double Blind Week 16 LOCF | Placebo | −4.70 | 0.43 | | | | |
| | TC-5214 | −8.60 | 0.43 | | | | |
| | | | | 3.90 | 2.70 | 5.10 | <0.0001 |
| Double Blind Week 18 LOCF | Placebo | 0.19 | 0.29 | | | | |
| (Follow-up) | TC-5214 | −0.65 | 0.28 | | | | |
| | | | | 0.84 | 0.03 | 1.66 | 0.0442 |

Definitions: LOCF = Last Observation Carried Forward; CI = Confidence Interval

With regard to HAMD Anxiety/Somatization, as shown in Table 7, the TC-5214 group had statistically significant decreases (improvements) from baseline in HAMD anxiety/somatization subscale score compared with the placebo group for both the ITT and PP populations.

TABLE 7

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −2.65 | 0.23 | | | | |
| | TC-5214 | −4.58 | 0.23 | | | | |
| | | | | 1.93 | 1.29 | 2.57 | <0.0001 |
| Double Blind Week 18 LOCF | Placebo | −0.14 | 0.12 | | | | |
| (Follow-up) | TC-5214 | −0.14 | 0.12 | | | | |
| | | | | −0.00 | −0.35 | 0.35 | 0.9948 |
| PP Population | | | | | | | |
| Double Blind Week 16 LOCF | Placebo | −3.01 | 0.25 | | | | |
| | TC-5214 | −4.93 | 0.25 | | | | |
| | | | | 1.92 | 1.24 | 2.60 | <0.0001 |
| Double Blind Week 18 LOCF | Placebo | −0.15 | 0.14 | | | | |
| (Follow-up) | TC-5214 | −0.16 | 0.14 | | | | |
| | | | | 0.01 | −0.40 | 0.42 | 0.9691 |

Definitions: LOCF = Last Observation Carried Forward; CI = Confidence Interval

With regard to MADRS, as shown in Table 8, the TC-5214 group had statistically significant decreases (improvements) from baseline in MADRS total score compared with the placebo group for both the ITT and PP populations.

TABLE 8

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −9.72 | 0.88 | | | | |
| | TC-5214 | −17.26 | 0.88 | | | | |
| | | | | 7.54 | 5.10 | 9.98 | <0.0001 |
| Double Blind Week 18 LOCF | Placebo | −1.41 | 0.38 | | | | |
| (Follow-up) | TC-5214 | −0.87 | 0.38 | | | | |
| | | | | −0.54 | −1.63 | 0.55 | 0.3343 |
| PP Population | | | | | | | |
| Double Blind Week 16 LOCF | Placebo | −11.38 | 0.89 | | | | |
| | TC-5214 | −19.34 | 0.88 | | | | |
| | | | | 7.97 | 5.52 | 10.42 | <0.0001 |
| Double Blind Week 18 LOCF | Placebo | −1.29 | 0.44 | | | | |
| (Follow-up) | TC-5214 | −1.10 | 0.44 | | | | |
| | | | | −0.20 | −1.45 | 1.06 | 0.7608 |

Definitions: LOCF = Last Observation Carried Forward; CI = Confidence Interval

With regard to MADRS Proportion of Remission, the first endpoint was the proportion of subjects assesses to be in remission as defined by a MADRS score of ≦10 at Week 16. The second endpoint was the proportion of subjects assesses to be in remission as defined by a MADRS score of ≦at Week 16. Fisher's Exact Test was used to compare the proportions of subjects with a remission between the two treatment groups. Tables 9 and 10, respectively, show the secondary efficacy analysis for each group.

TABLE 9

| | | | | | | P-Value |
|---|---|---|---|---|---|---|
| | | | | | | (Fisher |
| ITT | | Placebo | | TC-5214 | | Exact |
| Population | Parameter | (N = 132) | | (N = 133) | | Test) |
| Double Blind | Subjects | 104 | (79%) | 64 | (48%) | |
| Week 16 | without | | | | | |
| | Remission | | | | | |
| | Subjects with | 28 | (21%) | 69 | (52%) | |
| | Remission | | | | | |
| | P-Value for | | | | | <0.0001 |
| | TC-5214 vs | | | | | |
| | Placebo | | | | | |
| Double Blind | Subjects | 95 | (72%) | 60 | (45%) | |
| Week 18 | without | | | | | |
| (Follow-up) | Remission | | | | | |
| | Subjects with | 37 | (28%) | 73 | (55%) | |
| | Remission | | | | | |
| | P-Value for | | | | | <0.0001 |
| | TC-5214 vs | | | | | |
| | Placebo | | | | | |
| PP | | Placebo | | TC-5214 | | |
| Population | Parameter | (N = 112) | | (N = 113) | | |
| Double Blind | Subjects | 84 | (75%) | 46 | (41%) | |
| Week 16 | without | | | | | |
| | Remission | | | | | |
| | Subjects with | 28 | (25%) | 67 | (59%) | |
| | Remission | | | | | |
| | P-Value for | | | | | <0.0001 |
| | TC-5214 vs | | | | | |
| | Placebo | | | | | |
| Double Blind | Subjects | 75 | (67%) | 43 | (38%) | |
| Week 18 | without | | | | | |
| (Follow-up) | Remission | | | | | |
| | Subjects with | 37 | (33%) | 70 | (62%) | |
| | Remission | | | | | |
| | P-Value for | | | | | <0.0001 |
| | TC-5214 vs | | | | | |
| | Placebo | | | | | |

Note:
percentages are relative to the number of subjects in each treatment group

TABLE 10

| | | | | | | P-Value |
|---|---|---|---|---|---|---|
| | | | | | | (Fisher |
| ITT | | Placebo | | TC-5214 | | Exact |
| Population | Parameter | (N = 132) | | (N = 133) | | Test) |
| Double Blind | Subjects | 96 | (73%) | 56 | (42%) | |
| Week 16 | without | | | | | |
| | Remission | | | | | |
| | Subjects with | 36 | (27%) | 77 | (58%) | |
| | Remission | | | | | |
| | P-Value for | | | | | <0.0001 |
| | TC-5214 vs | | | | | |
| | Placebo | | | | | |
| Double Blind | Subjects | 89 | (67%) | 47 | (35%) | |
| Week 18 | without | | | | | |
| (Follow-up) | Remission | | | | | |
| | Subjects with | 43 | (33%) | 86 | (65%) | |
| | Remission | | | | | |
| | P-Value for | | | | | <0.0001 |
| | TC-5214 vs | | | | | |
| | Placebo | | | | | |
| PP | | Placebo | | TC-5214 | | |
| Population | Parameter | (N = 112) | | (N = 113) | | |
| Double Blind | Subjects | 76 | (68%) | 41 | (36%) | |
| Week 16 | without | | | | | |
| | Remission | | | | | |
| | Subjects with | 36 | (32%) | 72 | (64%) | |
| | Remission | | | | | |
| | P-Value for | | | | | <0.0001 |
| | TC-5214 vs | | | | | |
| | Placebo | | | | | |
| Double Blind | Subjects | 69 | (62%) | 32 | (28%) | |
| Week 18 | without | | | | | |
| (Follow-up) | Remission | | | | | |
| | Subjects with | 43 | (38%) | 81 | (72%) | |
| | Remission | | | | | |
| | P-Value for | | | | | <0.0001 |
| | TC-5214 vs | | | | | |
| | Placebo | | | | | |

Note:
percentages are relative to the number of subjects in each treatment group

As shown in Tables 9 and 10, the TC-5214 group had a higher proportion of subjects meeting the remission criteria compared with the placebo group as assessed by MADRS ≦10 or ≦12 for both the ITT and PP populations. All differences were statistically significant (P<0.0001) and demonstrate TC-5214 to be superior to placebo for MADRS remission rate.

Regarding MADRS Proportion of Responders, the endpoint was the proportion of subjects assessed to be responders as defined by MADRS reduction from baseline of ≧50%. Fisher's Exact Test was used to compare the proportions of responders between the two treatment groups.

TABLE 11

| | | | | | | P-Value |
|---|---|---|---|---|---|---|
| | | | | | | (Fisher |
| ITT | | Placebo | | TC-5214 | | Exact |
| Population | Parameter | (N = 132) | | (N = 133) | | Test) |
| Double Blind | Subjects | 85 | (64%) | 44 | (33%) | |
| Week 16 | without | | | | | |
| | Response | | | | | |
| | Subjects with | 47 | (36%) | 89 | (67%) | |
| | Response | | | | | |
| | P-Value for | | | | | <0.0001 |
| | TC-5214 vs | | | | | |
| | Placebo | | | | | |
| Double Blind | Subjects | 78 | (59%) | 39 | (29%) | |
| Week 18 | without | | | | | |
| (Follow-up) | Response | | | | | |
| | Subjects with | 54 | (41%) | 94 | (71%) | |
| | Response | | | | | |
| | P-Value for | | | | | <0.0001 |
| | TC-5214 vs | | | | | |
| | Placebo | | | | | |
| PP | | Placebo | | TC-5214 | | |
| Population | Parameter | (N = 112) | | (N = 113) | | |
| Double Blind | Subjects | 66 | (59%) | 29 | (26%) | |
| Week 16 | without | | | | | |
| | Response | | | | | |
| | Subjects with | 46 | (41%) | 84 | (74%) | |
| | Response | | | | | |
| | P-Value for | | | | | <0.0001 |
| | TC-5214 vs | | | | | |
| | Placebo | | | | | |
| Double Blind | Subjects | 59 | (53%) | 24 | (21%) | |
| Week 18 | without | | | | | |
| (Follow-up) | Response | | | | | |
| | Subjects with | 53 | (47%) | 89 | (79%) | |
| | Response | | | | | |
| | P-Value for | | | | | <0.0001 |
| | TC-5214 vs | | | | | |
| | Placebo | | | | | |

Note:
percentages are relative to the number of subjects in each treatment group

As shown in Table 11, the TC-5214 group had a higher proportion of responders compared with the placebo group as assessed by MADRS reduction from baseline ≧50% for both the ITT and PP populations. All differences were statistically significant (P<0.0001) and demonstrate TC-5214 to be superior to placebo regarding MADRS response rate.

Regarding HAMD-17 Proportion of Remission, the first endpoint was the proportion of subjects assessed to be in remission as defined by a HAMD-17 score of ≦7 at Week 16. The second endpoint was the proportion of subjects assessed to be in remission as defined by a HAMD-17 score of ≦10 at Week 16. Fisher's Exact Test was used to compare the proportions of subjects with a remission between the two treatment groups at each double-blind visit.

TABLE 12
HAMD-17 Remission (Score ≦7)

| | | | | | | P-Value |
|---|---|---|---|---|---|---|
| | | | | | | (Fisher |
| | | Placebo | | TC-5214 | | Exact |
| | Parameter | (N = 132) | | (N = 133) | | Test) |
| ITT Population | | | | | | |
| Double Blind Week 9 | Subjects without Remission | 132 | (100%) | 132 | (99%) | |
| | Subjects with Remission | 0 | (0%) | 1 | (1%) | |
| | P-Value for TC-5214 vs Placebo | | | | | 1.0000 |
| Double Blind Week 10 | Subjects without Remission | 131 | (99%) | 127 | (95%) | |
| | Subjects with Remission | 1 | (1%) | 6 | (5%) | |
| | P-Value for TC-5214 vs Placebo | | | | | 0.1200 |
| Double Blind Week 12 | Subjects without Remission | 126 | (95%) | 113 | (85%) | |
| | Subjects with Remission | 6 | (5%) | 20 | (15%) | |
| | P-Value for TC-5214 vs Placebo | | | | | 0.0063 |
| Double Blind Week 14 | Subjects without Remission | 117 | (89%) | 96 | (72%) | |
| | Subjects with Remission | 15 | (11%) | 37 | (28%) | |
| | P-Value for TC-5214 vs Placebo | | | | | 0.0010 |
| Double Blind Week 16 | Subjects without Remission | 107 | (81%) | 81 | (61%) | |
| | Subjects with Remission | 25 | (19%) | 52 | (39%) | |
| | P-Value for TC-5214 vs Placebo | | | | | 0.0004 |
| Double Blind Week 18 | Subjects without Remission | 100 | (76%) | 73 | (55%) | |
| (Follow-up) | Subjects with Remission | 32 | (24%) | 60 | (45%) | |
| | P-Value for TC-5214 vs Placebo | | | | | 0.0005 |
| PP Population | | | | | | |
| Double Blind Week 9 | Subjects without Remission | 112 | (100%) | 112 | (99%) | |
| | Subjects with Remission | 0 | (0%) | 1 | (1%) | |
| | P-Value for TC-5214 vs Placebo | | | | | 1.0000 |
| Double Blind Week 10 | Subjects without Remission | 111 | (99%) | 109 | (96%) | |
| | Subjects with Remission | 1 | (1%) | 4 | (4%) | |
| | P-Value for TC-5214 vs Placebo | | | | | 0.3694 |
| Double Blind Week 12 | Subjects without Remission | 106 | (95%) | 97 | (86%) | |
| | Subjects with Remission | 6 | (5%) | 16 | (14%) | |
| | P-Value for TC-5214 vs Placebo | | | | | 0.0414 |
| Double Blind Week 14 | Subjects without Remission | 97 | (87%) | 78 | (69%) | |
| | Subjects with Remission | 15 | (13%) | 35 | (31%) | |
| | P-Value for TC-5214 vs Placebo | | | | | 0.0021 |
| Double Blind Week 16 | Subjects without Remission | 87 | (78%) | 63 | (56%) | |
| | Subjects with Remission | 25 | (22%) | 50 | (44%) | |
| | P-Value for TC-5214 vs Placebo | | | | | 0.0006 |
| Double Blind Week 18 | Subjects without Remission | 80 | (71%) | 55 | (49%) | |
| (Follow-up) | Subjects with Remission | 32 | (29%) | 58 | (51%) | |
| | P-Value for TC-5214 vs Placebo | | | | | 0.0006 |

Note:
percentages are relative to the number of subjects in each treatment group

TABLE 13
HAMD-17 Remission (Score ≦10)

| | | | | | | P-Value |
|---|---|---|---|---|---|---|
| | | | | | | (Fisher |
| | | Placebo | | TC-5214 | | Exact |
| | Parameter | (N = 132) | | (N = 133) | | Test) |
| ITT Population | | | | | | |
| Double Blind Week 9 | Subjects without Remission | 127 | (96%) | 129 | (97%) | |
| | Subjects with Remission | 5 | (4%) | 4 | (3%) | |
| | P-Value for TC-5214 vs Placebo | | | | | 0.7492 |
| Double Blind Week 10 | Subjects without Remission | 123 | (93%) | 113 | (85%) | |
| | Subjects with Remission | 9 | (7%) | 20 | (15%) | |
| | P-Value for TC-5214 vs Placebo | | | | | 0.0476 |
| Double Blind Week 12 | Subjects without Remission | 118 | (89%) | 93 | (70%) | |
| | Subjects with Remission | 14 | (11%) | 40 | (30%) | |
| | P-Value for TC-5214 vs Placebo | | | | | 0.0001 |
| Double Blind Week 14 | Subjects without Remission | 102 | (77%) | 73 | (55%) | |
| | Subjects with Remission | 30 | (23%) | 60 | (45%) | |
| | P-Value for TC-5214 vs Placebo | | | | | 0.0002 |
| Double Blind Week 16 | Subjects without Remission | 94 | (71%) | 50 | (38%) | |
| | Subjects with Remission | 38 | (29%) | 83 | (62%) | |
| | P-Value for TC-5214 vs Placebo | | | | | <0.0001 |
| Double Blind Week 18 | Subjects without Remission | 84 | (64%) | 48 | (36%) | |
| (Follow-up) | Subjects with Remission | 48 | (36%) | 85 | (64%) | |
| | P-Value for TC-5214 vs Placebo | | | | | <0.0001 |
| PP Population | | | | | | |
| Double Blind Week 9 | Subjects without Remission | 107 | (96%) | 110 | (97%) | |
| | Subjects with Remission | 5 | (4%) | 3 | (3%) | |
| | P-Value for TC-5214 vs Placebo | | | | | 0.4989 |
| Double Blind Week 10 | Subjects without Remission | 103 | (92%) | 97 | (86%) | |
| | Subjects with Remission | 9 | (8%) | 16 | (14%) | |
| | P-Value for TC-5214 vs Placebo | | | | | 0.2025 |
| Double Blind Week 12 | Subjects without Remission | 98 | (88%) | 77 | (68%) | |
| | Subjects with Remission | 14 | (13%) | 36 | (32%) | |
| | P-Value for TC-5214 vs Placebo | | | | | 0.0007 |
| Double Blind Week 14 | Subjects without Remission | 84 | (75%) | 60 | (53%) | |
| | Subjects with Remission | 28 | (25%) | 53 | (47%) | |
| | P-Value for TC-5214 vs Placebo | | | | | 0.0008 |
| Double Blind Week 16 | Subjects without Remission | 76 | (68%) | 38 | (34%) | |
| | Subjects with Remission | 36 | (32%) | 75 | (66%) | |
| | P-Value for TC-5214 vs Placebo | | | | | <0.0001 |
| Double Blind Week 18 | Subjects without Remission | 67 | (60%) | 36 | (32%) | |
| (Follow-up) | Subjects with Remission | 45 | (40%) | 77 | (68%) | |
| | P-Value for TC-5214 vs Placebo | | | | | <0.0001 |

Note:
percentages are relative to the number of subjects in each treatment group

At almost every assessment week, the TC-5214 group had a higher proportion of subjects in remission compared with the placebo group as assessed by HAMD-17 for both the ITT and PP populations. Observations are Weeks 10 (score ≦10), 12, 14, 16, and 18 (both) provide statistically significant differences and demonstrate TC-5214 to be superior to placebo.

Regarding HAMD-17 Proportion of Responders, the endpoint was the proportion of subjects assessed to be responders as defined by HAMD-17 reduction from baseline ≧50%. Fisher's Exact Test was used to compare the proportions of responders between the two treatment groups at each double-blind visit.

TABLE 14

| | | | | | | P-Value |
|---|---|---|---|---|---|---|
| | | | | | | (Fisher |
| | | Placebo | | TC-5214 | | Exact |
| ITT Population | Parameter | (N = 132) | | (N = 133) | | Test) |
| Double Blind Week 9 | Subjects without Response | 128 | (97%) | 129 | (97%) | |
| | Subjects with Response | 4 | (3%) | 4 | (3%) | |
| | P-Value for TC-5214 vs Placebo | | | | | 1.0000 |
| Double Blind Week 10 | Subjects without Response | 125 | (95%) | 110 | (83%) | |
| | Subjects with Response | 7 | (5%) | 23 | (17%) | |
| | P-Value for TC-5214 vs Placebo | | | | | 0.0031 |
| Double Blind Week 12 | Subjects without Response | 113 | (86%) | 88 | (66%) | |
| | Subjects with Response | 19 | (14%) | 45 | (34%) | |
| | P-Value for TC-5214 vs Placebo | | | | | 0.0003 |
| Double Blind Week 14 | Subjects without Response | 99 | (75%) | 61 | (46%) | |
| | Subjects with Response | 33 | (25%) | 72 | (54%) | |
| | P-Value for TC-5214 vs Placebo | | | | | <0.0001 |
| Double Blind Week 16 | Subjects without Response | 89 | (67%) | 43 | (32%) | |
| | Subjects with Response | 43 | (33%) | 90 | (68%) | |
| | P-Value for TC-5214 vs Placebo | | | | | <0.0001 |
| Double Blind Week 18 | Subjects without Response | 82 | (62%) | 44 | (33%) | |
| (Follow-up) | Subjects with Response | 50 | (38%) | 89 | (67%) | |
| | P-Value for TC-5214 vs Placebo | | | | | <0.0001 |

TABLE 15

| | | | | | | P-Value |
|---|---|---|---|---|---|---|
| | | | | | | (Fisher |
| | | Placebo | | TC-5214 | | Exact |
| PP Population | Parameter | (N = 112) | | (N = 113) | | Test) |
| Double Blind Week 9 | Subjects without Response | 108 | (96%) | 110 | (97%) | |
| | Subjects with Response | 4 | (4%) | 3 | (3%) | |
| | P-Value for TC-5214 vs Placebo | | | | | 0.7216 |
| Double Blind Week 10 | Subjects without Response | 105 | (94%) | 93 | (82%) | |
| | Subjects with Response | 7 | (6%) | 20 | (18%) | |
| | P-Value for TC-5214 vs Placebo | | | | | 0.0127 |
| Double Blind Week 12 | Subjects without Response | 94 | (84%) | 73 | (65%) | |
| | Subjects with Response | 18 | (16%) | 40 | (35%) | |
| | P-Value for TC-5214 vs Placebo | | | | | 0.0013 |
| Double Blind Week 14 | Subjects without Response | 81 | (72%) | 46 | (41%) | |
| | Subjects with Response | 31 | (28%) | 67 | (59%) | |
| | P-Value for TC-5214 vs Placebo | | | | | <0.0001 |
| Double Blind Week 16 | Subjects without Response | 71 | (63%) | 29 | (26%) | |
| | Subjects with Response | 41 | (37%) | 84 | (74%) | |
| | P-Value for TC-5214 vs Placebo | | | | | <0.0001 |
| Double Blind Week 18 | Subjects without Response | 64 | (57%) | 30 | (27%) | |
| (Follow-up) | Subjects with Response | 48 | (43%) | 83 | (73%) | |
| | P-Value for TC-5214 vs Placebo | | | | | <0.0001 |

Note:
percentages are relative to the number of subjects in each treatment group

At almost every assessment week, the TC-5214 group had a higher proportion of responders compared with the placebo group as assessed by HAMD reduction from baseline of ≧50% for both the ITT (Table 14) and PP (Table 15) populations. Statistically significant differences in proportions of responders were observed at Weeks 10, 12, 14, 16, and 18 for the ITT and PP populations. The results demonstrate that TC-5214 was superior to placebo for each statistically significant comparison.

With regard to QIDS Proportion of Remission, the endpoint was the proportion of subjects assessed to be in remission as defined by a QIDS score of ≦5. Table 16 provides QIDS remission for the ITT population and Table 17 provides QIDS remission for the PP population.

TABLE 16

| | | | | | | P-Value |
|---|---|---|---|---|---|---|
| | | | | | | (Fisher |
| | | Placebo | | TC-5214 | | Exact |
| ITT Population | Parameter | (N = 132) | | (N = 133) | | Test) |
| Double Blind Week 10 | Subjects without Remission | 120 | (91%) | 114 | (86%) | |
| | Subjects with Remission | 12 | (9%) | 19 | (14%) | |
| | P-Value for TC-5214 vs Placebo | | | | | 0.2513 |
| Double Blind Week 12 | Subjects without Remission | 119 | (90%) | 108 | (81%) | |
| | Subjects with Remission | 13 | (10%) | 25 | (19%) | |
| | P-Value for TC-5214 vs Placebo | | | | | 0.0529 |
| Double Blind Week 14 | Subjects without Remission | 114 | (86%) | 88 | (66%) | |
| | Subjects with Remission | 18 | (14%) | 45 | (34%) | |
| | P-Value for TC-5214 vs Placebo | | | | | 0.0001 |
| Double Blind Week 16 | Subjects without Remission | 101 | (77%) | 73 | (55%) | |
| | Subjects with Remission | 31 | (23%) | 60 | (45%) | |
| | P-Value for TC-5214 vs Placebo | | | | | 0.0003 |
| Double Blind Week 18 | Subjects without Remission | 98 | (74%) | 58 | (44%) | |
| (Follow-up) | Subjects with Remission | 34 | (26%) | 75 | (56%) | |
| | P-Value for TC-5214 vs Placebo | | | | | <0.0001 |

TABLE 17

| | | | | P-Value |
|---|---|---|---|---|
| | | | | (Fisher |
| | | Placebo | TC-5214 | Exact |
| PP Population | Parameter | (N = 112) | (N = 113) | Test) |
| Double Blind Week 10 | Subjects without Remission | 102 (91%) | 96 (85%) | |
| | Subjects with Remission | 10 (9%) | 17 (15%) | |
| | P-Value for TC-5214 vs Placebo | | | 0.2180 |
| Double Blind Week 12 | Subjects without Remission | 100 (89%) | 90 (80%) | |
| | Subjects with Remission | 12 (11%) | 23 (20%) | |
| | P-Value for TC-5214 vs Placebo | | | 0.0649 |
| Double Blind Week 14 | Subjects without Remission | 95 (85%) | 74 (65%) | |
| | Subjects with Remission | 17 (15%) | 39 (35%) | |
| | P-Value for TC-5214 vs Placebo | | | 0.0011 |
| Double Blind Week 16 | Subjects without Remission | 82 (73%) | 58 (51%) | |
| | Subjects with Remission | 30 (27%) | 55 (49%) | |
| | P-Value for TC-5214 vs Placebo | | | 0.0009 |
| Double Blind Week 18 | Subjects without Remission | 79 (71%) | 44 (39%) | |
| (Follow-up) | Subjects with Remission | 33 (29%) | 69 (61%) | |
| | P-Value for TC-5214 vs Placebo | | | <0.0001 |

Note:
percentages are relative to the number of subjects in each treatment group

At every assessment week, the TC-5214 group had a higher proportion of subjects with remission compared with the placebo group as assessed by QIDS score of <5 for both the ITT and PP populations. Statistically significant differences in proportions of remission between the TC-5214 group and placebo group were observed at Weeks 14, 16, and 18 for the ITT and PP populations. The results demonstrate that TC-5214 was superior to placebo for each statistically significant comparison.

Regarding QIDS proportion of responders, the endpoint was the proportion of subjects assessed to be responders as defined by a QIDS reduction from baseline ≧50%. Tables 18 and 19 provide the results for the ITT and PP populations, respectively.

TABLE 18

| | | | | P-Value |
|---|---|---|---|---|
| | | | | (Fisher |
| | | Placebo | TC-5214 | Exact |
| ITT Population | Parameter | (N = 132) | (N = 133) | Test) |
| Double Blind Week 10 | Subjects without Response | 118 (89%) | 104 (78%) | |
| | Subjects with Response | 14 (11%) | 29 (22%) | |
| | P-Value for TC-5214 vs Placebo | | | 0.0190 |
| Double Blind Week 12 | Subjects without Response | 102 (77%) | 89 (67%) | |
| | Subjects with Response | 30 (23%) | 44 (33%) | |
| | P-Value for TC-5214 vs Placebo | | | 0.0749 |
| Double Blind Week 14 | Subjects without Response | 94 (71%) | 66 (50%) | |
| | Subjects with Response | 38 (29%) | 67 (50%) | |
| | P-Value for TC-5214 vs Placebo | | | 0.0004 |
| Double Blind Week 16 | Subjects without Response | 88 (67%) | 45 (34%) | |
| | Subjects with Response | 44 (33%) | 88 (66%) | |
| | P-Value for TC-5214 vs Placebo | | | <0.0001 |
| Double Blind Week 18 | Subjects without Response | 82 (62%) | 41 (31%) | |
| (Follow-up) | Subjects with Response | 50 (38%) | 92 (69%) | |
| | P-Value for TC-5214 vs Placebo | | | <0.0001 |

TABLE 19

| | | | | P-Value |
|---|---|---|---|---|
| | | | | (Fisher |
| | | Placebo | TC-5214 | Exact |
| PP Population | Parameter | (N = 112) | (N = 113) | Test) |
| Double Blind Week 10 | Subjects without Response | 99 (88%) | 88 (78%) | |
| | Subjects with Response | 13 (12%) | 25 (22%) | |
| | P-Value for TC-5214 vs Placebo | | | 0.0494 |
| Double Blind Week 12 | Subjects without Response | 85 (76%) | 73 (65%) | |
| | Subjects with Response | 27 (24%) | 40 (35%) | |
| | P-Value for TC-5214 vs Placebo | | | 0.0801 |
| Double Blind Week 14 | Subjects without Response | 78 (70%) | 54 (48%) | |
| | Subjects with Response | 34 (30%) | 59 (52%) | |
| | P-Value for TC-5214 vs Placebo | | | 0.0011 |
| Double Blind Week 16 | Subjects without Response | 72 (64%) | 33 (29%) | |
| | Subjects with Response | 40 (36%) | 80 (71%) | |
| | P-Value for TC-5214 vs Placebo | | | <0.0001 |
| Double Blind Week 18 | Subjects without Response | 66 (59%) | 29 (26%) | |
| (Follow-up) | Subjects with Response | 46 (41%) | 84 (74%) | |
| | P-Value for TC-5214 vs Placebo | | | <0.0001 |

Note:
percentages are relative to the number of subjects in each treatment group

At every assessment week, the TC-5214 group had a higher proportion of responders compared with the placebo group as assessed by QIDS reduction from baseline of ≧50% for both the ITT and PP populations. Statistically significant differences in proportions of response between the TC-5214 group and the placebo group were observed at Weeks 10, 14, 16, and 18 for both populations. The results demonstrate that TC-5214 was superior to placebo for each statistically significant comparison.

With regard to CGI-SI, the endpoint was the change from Week 8 to Week 16 in the CGI-SI. Changes from baseline (Week 8) to Week 16 in the CGI-SI were analyzed using ANCOVA. Table 20 provides secondary efficacy analysis results for CGI-SI.

TABLE 20

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −0.91 | 0.10 | | | | |
| | TC-5214 | −1.79 | 0.10 | | | | |
| | | | | 0.87 | 0.61 | 1.14 | <0.0001 |
| Double Blind Week 18 LOCF | Placebo | −0.04 | 0.05 | | | | |
| (Follow-up) | TC-5214 | −0.05 | 0.05 | | | | |
| | | | | 0.01 | −0.12 | 0.14 | 0.8929 |
| PP Population | | | | | | | |
| Double Blind Week 16 LOCF | Placebo | −1.08 | 0.10 | | | | |
| | TC-5214 | −2.00 | 0.10 | | | | |
| | | | | 0.92 | 0.65 | 1.20 | <0.0001 |
| Double Blind Week 18 LOCF | Placebo | −0.02 | 0.05 | | | | |
| (Follow-up) | TC-5214 | −0.07 | 0.05 | | | | |
| | | | | 0.05 | −0.10 | 0.20 | 0.5278 |

Definitions: LOCF = Last Observation Carried Forward; CI = Confidence Interval

As shown in Table 20, the TC-5214 group had statistically significant decreases (improvements) from baseline in CGI-SI score compared with the placebo group for both the ITT and PP populations.

With regard to CGI-I, the endpoint was the change from Week 8 (double-blind baseline) to Weeks 16 and 18 in the CGI-I. Changes were analyzed using ANCOVA. In addition, an exploratory analysis was performed that evaluated CGI-I at all visit weeks. Table 21 provides secondary efficacy analysis results for CGI-I.

TABLE 21

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | 2.70 | 0.09 | | | | |
| | TC-5214 | 1.91 | 0.09 | | | | |
| | | | | 0.79 | 0.54 | 1.04 | <0.0001 |
| Double Blind Week 18 LOCF | Placebo | 2.61 | 0.10 | | | | |
| (Follow-up) | TC-5214 | 1.90 | 0.10 | | | | |
| | | | | 0.71 | 0.45 | 0.98 | <0.0001 |
| PP Population | | | | | | | |
| Double Blind Week 16 LOCF | Placebo | 2.63 | 0.10 | | | | |
| | TC-5214 | 1.84 | 0.10 | | | | |
| | | | | 0.78 | 0.52 | 1.05 | <0.0001 |
| Double Blind Week 18 LOCF | Placebo | 2.54 | 0.10 | | | | |
| (Follow-up) | TC-5214 | 1.83 | 0.10 | | | | |
| | | | | 0.71 | 0.43 | 1.00 | <0.0001 |

Definitions: LOCF = Last Observation Carried Forward; CI = Confidence Interval

As shown in Table 21, the TC-5214 group had statistically significant decreases (improvements) from baseline in CGI-I score compared with the placebo group for both the ITT and PP populations at Week 16 and at Week 18.

For SDS, the endpoint was the change from Week 8 (double-blind baseline) to Week 16 of the SDS. Change was analyzed using ANCOVA. The secondary efficacy analysis results are provided in Table 22.

TABLE 22

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −4.58 | 0.53 | | | | |
| | TC-5214 | −9.18 | 0.53 | | | | |
| | | | | 4.60 | 3.13 | 6.08 | <0.0001 |
| Double Blind Week 18 LOCF | Placebo | −0.69 | 0.24 | | | | |
| (Follow-up) | TC-5214 | −0.46 | 0.23 | | | | |
| PP Population | | | | −0.23 | −0.90 | 0.45 | 0.5094 |
| Double Blind Week 16 LOCF | Placebo | −5.35 | 0.55 | | | | |
| | TC-5214 | −10.21 | 0.54 | | | | |
| | | | | 4.86 | 3.35 | 6.37 | <0.0001 |
| Double Blind Week 18 LOCF | Placebo | −0.64 | 0.26 | | | | |
| (Follow-up) | TC-5214 | −0.44 | 0.25 | | | | |
| | | | | −0.20 | −0.93 | 0.53 | 0.5885 |

Definitions: LOCF = Last Observation Carried Forward; CI = Confidence Interval

As shown in Table 22, the TC-5214 group had statistically significant decreases (improvements) from baseline in SDS score compared with the placebo group for both the ITT and PP populations. For SDS individual items (Work-School; Social Life; and Life-Home Responsibilities), the TC-5214 group had statistically significant decreases (improvements) from baseline compared with the placebo group for both the ITT and PP populations.

For SIS, the endpoint was the change from Week 8 (double-blind baseline) to Week 16 of the SIS. Changes were analyzed using ANCOVA. Each SIS item score was also analyzed using ANCOVA. The secondary efficacy analysis results are provided in Table 23.

TABLE 23

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| Double Blind Week 16 LOCF | Placebo | −9.45 | 1.12 | | | | |
| | TC-5214 | −18.21 | 1.11 | | | | |
| | | | | 8.76 | 5.67 | 11.85 | <0.0001 |
| Double Blind Week 18 LOCF | Placebo | −1.19 | 0.44 | | | | |
| (Follow-up) | TC-5214 | −0.68 | 0.44 | | | | |
| | | | | −0.51 | −1.75 | 0.74 | 0.4269 |
| PP Population | | | | | | | |
| Double Blind Week 16 LOCF | Placebo | −9.92 | 1.17 | | | | |
| | TC-5214 | −19.96 | 1.17 | | | | |
| | | | | 10.04 | 6.79 | 13.28 | <0.0001 |
| Double Blind Week 18 LOCF | Placebo | −1.11 | 0.48 | | | | |
| (Follow-up) | TC-5214 | −0.77 | 0.48 | | | | |
| | | | | −0.34 | −1.73 | 1.05 | 0.6334 |

Definitions: LOCF = Last Observation Carried Forward; CI = Confidence Interval

As shown in Table 23, the TC-5214 group had statistically significant decreases (improvements) from baseline in SIS total score compared with the placebo group for both the ITT and PP populations. SIS individual items, including Anger with others; Anger with self; Edginess; Frustration; Irritability; Moodiness; and Temper, were also assessed and tables for each are provided herein in respective order.

TABLE 24
Anger With Others

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS ANGER WITH OTHERS
ITT POPULATION

| | | | | | | | |
|---|---|---|---|---|---|---|---|
| Double Blind Week 16 LOCF | Placebo | −1.34 | 0.18 | | | | |
| | TC5214 | −2.56 | 0.18 | | | | |
| | | | | 1.22 | 0.73 | 1.71 | <0.0001 |
| Double Blind Week 18 LOCF | Placebo | −0.06 | 0.07 | | | | |
| (Follow-up) | TC5214 | −0.07 | 0.07 | | | | |
| | | | | 0.01 | −0.19 | 0.22 | 0.9180 |

ANALYSIS OF CHANGE FROM DB BASELINE AND CHANGE FROM WEEK 16 FOR SIS ANGER WITH OTHERS
PP POPULATION

| | | | | | | | |
|---|---|---|---|---|---|---|---|
| Double Blind Week 16 LOCF | Placebo | −1.35 | 0.18 | | | | |
| | TC5214 | −2.77 | 0.18 | | | | |
| | | | | 1.42 | 0.92 | 1.93 | <0.0001 |
| Double Blind Week 18 LOCF | Placebo | −0.07 | 0.08 | | | | |
| (Follow-up) | TC5214 | −0.10 | 0.08 | | | | |
| | | | | 0.03 | −0.21 | 0.27 | 0.8061 |

TABLE 25
Anger With Self

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS ANGER WITH SELF
ITT POPULATION

| | | | | | | | |
|---|---|---|---|---|---|---|---|
| Double Blind Week 16 LOCF | Placebo | −1.28 | 0.16 | | | | |
| | TC5214 | −2.29 | 0.16 | | | | |
| | | | | 1.01 | 0.57 | 1.44 | <0.0001 |
| Double Blind Week 18 LOCF | Placebo | 0.02 | 0.08 | | | | |
| (Follow-up) | TC5214 | −0.07 | 0.08 | | | | |
| | | | | 0.09 | −0.14 | 0.32 | 0.4499 |

ANALYSIS OF CHANGE FROM DB BASELINE AND CHANGE FROM WEEK 16 FOR SIS ANGER WITH SELF
PP POPULATION

| | | | | | | | |
|---|---|---|---|---|---|---|---|
| Double Blind Week 16 LOCF | Placebo | −1.41 | 0.17 | | | | |
| | TC5214 | −2.54 | 0.17 | | | | |
| | | | | 1.13 | 0.67 | 1.59 | <0.0001 |
| Double Blind Week 18 LOCF | Placebo | 0.02 | 0.10 | | | | |
| (Follow-up) | TC5214 | −0.09 | 0.10 | | | | |
| | | | | 0.11 | −0.16 | 0.38 | 0.4310 |

TABLE 26
Edginess

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS EDGINESS
ITT POPULATION

| | | | | | | | |
|---|---|---|---|---|---|---|---|
| Double Blind Week 16 LOCF | Placebo | −1.41 | 0.17 | | | | |
| | TC5214 | −2.55 | 0.17 | | | | |
| | | | | 1.14 | 0.66 | 1.61 | <0.0001 |
| Double Blind Week 18 LOCF | Placebo | −0.23 | 0.08 | | | | |
| (Follow-up) | TC5214 | −0.24 | 0.08 | | | | |
| | | | | 0.01 | −0.22 | 0.23 | 0.9441 |

ANALYSIS OF CHANGE FROM DB BASELINE AND CHANGE FROM WEEK 16 FOR SIS EDGINESS
PP POPULATION

| | | | | | | | |
|---|---|---|---|---|---|---|---|
| Double Blind Week 16 LOCF | Placebo | −1.46 | 0.18 | | | | |
| | TC5214 | −2.81 | 0.18 | | | | |
| | | | | 1.36 | 0.86 | 1.85 | <0.0001 |
| Double Blind Week 18 LOCF | Placebo | −0.22 | 0.09 | | | | |
| (Follow-up) | TC5214 | −0.27 | 0.09 | | | | |
| | | | | 0.05 | −0.20 | 0.30 | 0.6903 |

TABLE 27
Frustration

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS FRUSTRATION
ITT POPULATION

| | | | | | | | |
|---|---|---|---|---|---|---|---|
| Double Blind Week 16 LOCF | Placebo | −1.33 | 0.17 | | | | |
| | TC5214 | −2.74 | 0.17 | | | | |
| | | | | 1.42 | 0.94 | 1.90 | <0.0001 |
| Double Blind Week 18 LOCF | Placebo | −0.15 | 0.09 | | | | |
| (Follow-up) | TC5214 | −0.09 | 0.08 | | | | |
| | | | | −0.06 | −0.30 | 0.18 | 0.6245 |

ANALYSIS OF CHANGE FROM DB BASELINE AND CHANGE FROM WEEK 16 FOR SIS FRUSTRATION
PP POPULATION

| | | | | | | | |
|---|---|---|---|---|---|---|---|
| Double Blind Week 16 LOCF | Placebo | −1.41 | 0.18 | | | | |
| | TC5214 | −3.00 | 0.18 | | | | |
| | | | | 1.59 | 1.08 | 2.09 | <0.0001 |
| Double Blind Week 18 LOCF | Placebo | −0.13 | 0.09 | | | | |
| (Follow-up) | TC5214 | −0.09 | 0.09 | | | | |
| | | | | −0.04 | −0.30 | 0.22 | 0.7799 |

TABLE 28
Irritability

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS IRRITABILITY
ITT POPULATION

| | | | | | | | |
|---|---|---|---|---|---|---|---|
| Double Blind Week 16 LOCF | Placebo | −1.36 | 0.17 | | | | |
| | TC5214 | −2.74 | 0.17 | | | | |
| | | | | 1.38 | 0.90 | 1.87 | <0.0001 |
| Double Blind Week 18 LOCF | Placebo | −0.23 | 0.08 | | | | |
| (Follow-up) | TC5214 | −0.14 | 0.08 | | | | |
| | | | | −0.08 | −0.31 | 0.15 | 0.4914 |

ANALYSIS OF CHANGE FROM DB BASELINE AND CHANGE FROM WEEK 16 FOR SIS IRRITABILITY
PP POPULATION

| | | | | | | | |
|---|---|---|---|---|---|---|---|
| Double Blind Week 16 LOCF | Placebo | −1.42 | 0.18 | | | | |
| | TC5214 | −3.00 | 0.18 | | | | |
| | | | | 1.58 | 1.09 | 2.08 | <0.0001 |
| Double Blind Week 18 LOCF | Placebo | −0.21 | 0.09 | | | | |
| (Follow-up) | TC5214 | −0.14 | 0.09 | | | | |
| | | | | −0.07 | −0.32 | 0.19 | 0.6091 |

TABLE 29
Moodiness

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS MOODINESS
ITT POPULATION

| | | | | | | | |
|---|---|---|---|---|---|---|---|
| Double Blind Week 16 LOCF | Placebo | −1.49 | 0.18 | | | | |
| | TC5214 | −3.00 | 0.18 | | | | |
| | | | | 1.51 | 1.01 | 2.01 | <0.0001 |
| Double Blind Week 18 LOCF | Placebo | −0.18 | 0.07 | | | | |
| (Follow-up) | TC5214 | −0.13 | 0.07 | | | | |
| | | | | −0.05 | −0.26 | 0.17 | 0.6742 |

ANALYSIS OF CHANGE FROM DB BASELINE AND CHANGE FROM WEEK 16 FOR SIS MOODINESS
PP POPULATION

| | | | | | | | |
|---|---|---|---|---|---|---|---|
| Double Blind Week 16 LOCF | Placebo | −1.63 | 0.19 | | | | |
| | TC5214 | −3.34 | 0.19 | | | | |
| | | | | 1.71 | 1.18 | 2.23 | <0.0001 |
| Double Blind Week 18 LOCF | Placebo | −0.14 | 0.08 | | | | |
| (Follow-up) | TC5214 | −0.13 | 0.08 | | | | |
| | | | | −0.01 | −0.24 | 0.22 | 0.9427 |

TABLE 30
Temper

| | | | | | | | |
|---|---|---|---|---|---|---|---|
| | | | | Mean | | | |
| | | | | Difference | 95% | 95% | |
| | | | Standard | between | CL for | CL for | |
| | | | Error of | TC-5214 | Mean | Mean | |
| | | Adjusted | Adjusted | and | Difference | Difference | |
| Visit | Treatment | Mean | Mean | Placebo | Lower | Upper | P-Value |

ANALYSIS OF CHANGE FROM DB BASELINE AND CHANGE FROM WEEK 16 FOR SIS TEMPER
ITT POPULATION

| | | | | | | | |
|---|---|---|---|---|---|---|---|
| Double Blind Week 16 LOCF | Placebo | −1.18 | 0.17 | | | | |
| | TC5214 | −2.37 | 0.17 | | | | |
| | | | | 1.19 | 0.73 | 1.66 | <0.0001 |
| Double Blind Week 18 LOCF | Placebo | −0.18 | 0.08 | | | | |
| (Follow-up) | TC5214 | −0.11 | 0.08 | | | | |
| | | | | −0.07 | −0.30 | 0.15 | 0.5277 |

ANALYSIS OF CHANGE FROM DB BASELINE AND CHANGE FROM WEEK 16 FOR SIS TEMPER
PP POPULATION

| | | | | | | | |
|---|---|---|---|---|---|---|---|
| Double Blind Week 16 LOCF | Placebo | −1.19 | 0.17 | | | | |
| | TC5214 | −2.55 | 0.17 | | | | |
| | | | | 1.36 | 0.88 | 1.84 | <0.0001 |
| Double Blind Week 18 LOCF | Placebo | −0.15 | 0.09 | | | | |
| (Follow-up) | TC5214 | −0.14 | 0.09 | | | | |
| | | | | −0.00 | −0.25 | 0.24 | 0.9738 |

For each SIS individual item, the TC-5214 group had statistically significant decreases (improvements) from baseline compared with the placebo group for both the ITT and PP populations.

With regard to SGI-Cog, the endpoint was the Week 16 results for the SGI-Cog. The Week 16 SGI-Cog composite score and each scale were analyzed at Week 16 using ANOVA. Table 31 provides the secondary efficacy analysis results for SGI-Cog.

TABLE 31

| | | | | Mean | | | |
|---|---|---|---|---|---|---|---|
| | | | | Difference | | | |
| | | | Standard | between | 95% CI | 95% CI | |
| | | | Error of | TC-5214 | for Mean | for Mean | |
| | | Adjusted | Adjusted | and | Difference | Difference | |
| | Treatment | Mean | Mean | Placebo | Lower | Upper | P-Value |
| ITT Population | | | | | | | |
| TOTAL | Placebo | 8.66 | 0.27 | | | | |
| Double Blind Week 16 | TC-5214 | 6.52 | 0.27 | | | | |
| | | | | 2.15 | 1.41 | 2.89 | <0.0001 |
| MEMORY | Placebo | 2.90 | 0.09 | | | | |
| Double Blind Week 16 | TC-5214 | 2.23 | 0.09 | | | | |
| | | | | 0.67 | 0.42 | 0.93 | <0.0001 |
| ATTENTION | Placebo | 2.85 | 0.09 | | | | |
| Double Blind Week 16 | TC-5214 | 2.13 | 0.09 | | | | |
| | | | | 0.72 | 0.47 | 0.96 | <0.0001 |
| SPEED OF THINKING | Placebo | 2.92 | 0.09 | | | | |
| Double Blind Week 16 | TC-5214 | 2.16 | 0.09 | | | | |
| | | | | 0.76 | 0.50 | 1.02 | <0.0001 |
| PP Population | | | | | | | |
| TOTAL | Placebo | 8.48 | 0.26 | | | | |
| Double Blind Week 16 | TC-5214 | 6.23 | 0.26 | | | | |
| | | | | 2.25 | 1.53 | 2.97 | <0.0001 |
| MEMORY | Placebo | 2.83 | 0.09 | | | | |
| Double Blind Week 16 | TC-5214 | 2.15 | 0.09 | | | | |
| | | | | 0.68 | 0.42 | 0.93 | <0.0001 |
| ATTENTION | Placebo | 2.79 | 0.09 | | | | |
| Double Blind Week 16 | TC-5214 | 2.03 | 0.09 | | | | |
| | | | | 0.76 | 0.52 | 1.00 | <0.0001 |
| SPEED OF THINKING | Placebo | 2.87 | 0.09 | | | | |
| Double Blind Week 16 | TC-5214 | 2.05 | 0.09 | | | | |
| | | | | 0.81 | 0.56 | 1.07 | <0.0001 |

Definitions: CI = Confidence Interval

As shown in Table 31, the TC-5214 group had statistically significant lower Total SGI-Cog scores at Week 16 compared with the placebo group for both the ITT and PP populations.

An exploratory analysis assessed the differences in the treatment groups from Week 8 (double-blind baseline) to Week 16 for each individual HAMD-17 factor score. A summary is provided in Table 32.

TABLE 32

| | | Adjusted | | |
|---|---|---|---|---|
| | | Mean Change | | |
| | | TC-5214—Placebo | Effect | |
| Item | | (SD) | Size | P-Value |
| 1. | Depression-Mood | −0.69 (0.86) | 0.80 | <0.0001 |
| 2. | Feelings of Guilt | −0.43 (0.72) | 0.60 | <0.0001 |
| 3. | Suicide | −0.07 (0.40) | 0.18 | 0.1534 |
| 4. | Early Insomnia | −0.41 (0.64) | 0.64 | <0.0001 |
| 5. | Middle Insomnia | −0.42 (0.64) | 0.66 | <0.0001 |
| 6. | Late Insomnia | −0.35 (0.63) | 0.56 | <0.0001 |
| 7. | Work and Activities | −0.49 (0.86) | 0.57 | <0.0001 |
| 8. | Retardation | −0.31 (0.59) | 0.53 | <0.0001 |
| 9. | Agitation | −0.40 (0.68) | 0.59 | <0.0001 |
| 10. | Psychic Anxiety | −0.59 (0.77) | 0.77 | <0.0001 |
| 11. | Somatic Symptoms: | −0.41 (0.70) | 0.59 | <0.0001 |
| | Anxiety | | | |
| 12. | Somatic Symptoms: | −0.23 (0.52) | 0.44 | 0.0005 |
| | GI | | | |
| 13. | Somatic Symptoms: | −0.24 (0.66) | 0.36 | 0.0033 |
| | General | | | |
| 14. | Somatic Symptoms: | −0.34 (0.62) | 0.55 | <0.0001 |
| | Genital | | | |
| 15. | Hypochondriasis | −0.48 (0.74) | 0.65 | <0.0001 |
| 16. | Weight Loss | −0.20 (0.53) | 0.38 | 0.0024 |
| 17. | Insight | 0.03 (0.31) | 0.10 | 0.5023 |

Definitions: LOCF = Last Observation Carried Forward; SD = Standard Deviation; GI = Gastrointestinal

As shown in Table 32, the TC-5214 group had statistically significant decreases (improvements) from baseline in each HAMD-17 subscale score compared with the placebo group for the ITT population, with the exception of the Suicide and Insight subscales.

The exploratory analysis assessed the difference between the treatment groups in HAMD-17 total score at Weeks 8, 9, 10, 12, 14, and 16 for the ITT population. Graphic Table 33 shows the early onset of effect for HAMD-17 raw score.

Graphic Table 33
As shown, the TC-5214 group had statistically significant decreases (improvements) from baseline as compared with the placebo group at Weeks 10, 12, 14, and 16.

The exploratory analysis assessed the differences in the treatment groups in change from Week 8 (double-blind baseline) to Week 16 for each individual MADRS factor score. A summary is provided in Table 34.

TABLE 34

| | | Adjusted | | |
|---|---|---|---|---|
| | | Mean Change | | |
| | | TC-5214—Placebo | Effect | |
| Item | | (SD) | Size | P-Value |
| 1. | Apparent Sadness | −0.88 (1.26) | 0.70 | <0.0001 |
| 2. | Reported Sadness | −0.88 (1.28) | 0.69 | <0.0001 |
| 3. | Inner Tension | −0.79 (1.13) | 0.70 | <0.0001 |
| 4. | Reduced Sleep | −0.99 (1.40) | 0.71 | <0.0001 |
| 5. | Reduced Appetite | −0.76 (1.30) | 0.58 | <0.0001 |
| 6. | Concentration | −0.70 (1.20) | 0.58 | <0.0001 |
| | Difficulties | | | |
| 7. | Lassitude | −0.75 (1.20) | 0.63 | <0.0001 |
| 8. | Inability to Feel | −0.76 (1.25) | 0.61 | <0.0001 |
| 9. | Pessimistic Thoughts | −0.74 (1.18) | 0.63 | <0.0001 |
| 10. | Suicidal Thoughts | −0.29 (0.61) | 0.48 | 0.0002 |
| | Total Score | −7.54 (10.13) | 0.74 | <0.0001 |

Definitions: LOCF = Last Observation Carried Forward; SD = Standard Deviation; GI = Gastrointestinal

As shown, the TC-5214 group had statistically significant decreases (improvements) from baseline in each MADRS subscale score compared with the placebo group for the ITT population.

The specific pharmacological responses observed may vary according to and depending on the particular active compound, including a particular salt form, selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

Reference to methods and procedures is made to the following Example Protocol

### Example Protocol

1. A method of reducing one or more symptoms of depression to a subject in need thereof by administering exo-S-mecamylamine substantially free of exo-R-mecamylamine.

2. The method of claim 1, wherein the reduction comprises eliminating one or more symptoms of.

3. The method of claim 1, wherein the reduction comprises increasing remission or response rate from one or more symptoms of depression.

4. The method of claim 1, wherein the reduction comprises treating one or more symptoms of depression to remission or response.

5. The method of claim 1, wherein the one or more symptoms are related to one or more of
a. Cognition;
b. Attention;
c. Memory; and
d. Speed of thinking,
wherein measurement is made by a Subject Global Impression—Cognition scale change from baseline.

6. The method of claim 1, wherein the one or more symptom is measured by one or more of HAM-D, Sheehan Disability Scale, Sheehan Irritability Scale, MADRS, IDS, or QIDS.

7. The method of claim 1, wherein the reduction comprises improving cognitive function in a depressed subject.

8. A method for decreasing irritability in a subject by administering exo-S-mecamylamine substantially free of exo-R-mecamylamine.

9. The method of claim 8, wherein the subject is a depressed subject.

10. The method of claim 8, wherein the exo-S-mecamylamine substantially free of exo-R-mecamylamine is administered to subjects that are partial responders or non-responders to at least one other treatment.

11. The method of claim 10, wherein the other treatment was an antidepressant or antipsychotic.

12. The method of claim 11, wherein the anti-depressant is an SSRI or an SNRI.

13. The method of claim 1, wherein the dose of exo-S-mecamylamine substantially free of exo-R-mecamylamine is 1 mg or 2 mg daily.

14. The method of claim 1 wherein the rate of onset is about 2 weeks.

15. The method of claim 1, wherein the exo-S-mecamylamine substantially free of exo-R-mecamylamine maintains a sustained effect of at least 8 weeks.

16. The method of claim 1, wherein the administration of exo-S-mecamylamine substantially free of exo-R-mecamylamine provides a higher therapeutic index over conventional therapy.

17. The method of claim 1, wherein the administration of exo-S-mecamylamine substantially free of exo-R-mecamylamine provides a higher therapeutic index over first-line therapy.

18. The method of claim 1, wherein the subject is diagnosed with depression characterized by one or more of cognitive deficit, attention deficit, irritability, anxiety, disability, decreased quality of life, or memory deficit.

19. A combination comprising:
a. exo-S-mecamylamine substantially free of exo-R-mecamylamine; and
b. one or more antidepressant or antipsychotic.

20. A kit comprising:
a. the combination of claim 19, either separate or unitary;
b. and
instruction regarding a treatment regimen to treat, delay onset, increase remission or response rate, or delay progression progression of one or more symptoms of depression.

21. A pharmaceutical composition comprising:
a. the combination of claim 19
b. and
one or more pharmaceutically acceptable carrier.

22. The combination of claim 19, wherein the one or more antidepressant or antipsychotic is an SSRI or and SNRI.

23. The kit of claim 20, wherein the one or more symptoms is selected from cognitive deficit, attention deficit, irritability, anxiety, disability, decreased quality of life, or memory deficit.

24.-59. (canceled)
